(19) United States
(12) Patent Application Publication
(10) Pub. No.: US 2024/0428939 A1
(43) Pub. Date: Dec. 26, 2024

(54) SYSTEMS AND METHODS FOR EVALUATION OF MITOTIC EVENTS USING MACHINE-LEARNING

(71) Applicant: Volastra Therapeutics, Inc., New York, NY (US)

(72) Inventors: Akanksha VERMA, New York, NY (US); Christina ENG, Leonia, NJ (US); Sarah BETTIGOLE, New York, NY (US); Rahul GOPALKRISHNAN, Redmond, WA (US); Kristen SEVERSON, Redmond, WA (US); Nicolo FUSI, Redmond, WA (US); Philip ROSENFIELD, Cambridge, MA (US); Zongliang JI, Toronto (CA); Aliasghar MORTAZI, New York, NY (US)

(73) Assignee: Volastra Therapeutics, Inc., New York, NY (US)

(21) Appl. No.: 18/693,483

(22) PCT Filed: Sep. 21, 2022
(86) PCT No.: PCT/US2022/044287

§ 371 Date: Mar. 19, 2024

Related U.S. Application Data
(60) Provisional application No. 63/247,158, filed on Sep. 22, 2021.
(60) Provisional application No. 63/402,854, filed on Aug. 31, 2022.

(51) Int. Cl. G16H 50/20; G16H 30/40; G16H 50/70

(52) U.S. Cl. CPC G16H 50/20; G16H 30/40; G16H 50/70

(57) ABSTRACT

The present application provides methods and systems for detecting and quantifying chromosomal instability from histology images with machine-learning. Also described herein are methods for selecting treatments for a medical disease, by determining a chromosomal instability metric from histology images. The disclosed methods and systems may also be used to investigate disease progression and prognosis.

### CROSS REFERENCE TO RELATED APPLICATION

This application claims priority benefit of from U.S. Provisional Application No. 63/247,158, filed on Sep. 22, 2021, and U.S. Provisional Application No. 63/402,854, filed on Aug. 31, 2022, the contents of each of which are incorporated herein by reference in their entirety.

### FIELD

The present application relates to the methods and systems for determining chromosomal instability in a biological image, and use of the determined chromosomal instability for disease treatment and diagnosis.

### BACKGROUND

Chromosomal instability (CIN) results from errors in chromosome segregation during mitosis and contributes to poor prognosis, metastasis, and therapeutic resistance in human cancers. Identifying patient tumors with high levels of CIN can improve outcomes because CIN specific treatment options are available. Current methods to detect CIN in patient samples, such as bulk DNA sequencing, involve collecting additional data outside the standard of care, and can only infer CIN by detecting the mutations that result from CIN events rather than specifically detecting the CIN events (e.g., lagging chromosomes, polar chromosomes, abnormal mitosis, and/or the presence of micronuclei). Whole slide histology images are often acquired during tumor biopsy and/or extraction and display numerous cells undergoing mitosis, which may be indicative of a CIN state. Previous high throughput methods have used machine-learning techniques to identify cells undergoing mitosis. See, for example, Al-Janabi et al. 2013. “Evaluation of Mitotic Activity Index in Breast Cancer Using Whole Slide Digital Images”, PLOS ONE, 8 (12): e82576. However, these methods have not been able to identify or quantify specific abnormal mitotic events that more specifically characterize CIN and types of CIN, in an accurate and high throughput manner. In order to better diagnose and treat cancer patients, there is a need to directly quantify CIN specific mitotic events from readily available histology images with higher accuracy and efficiency. The present disclosure addressed these and other needs.

### BRIEF SUMMARY

Chromosomal instability refers to ongoing chromosome segregation errors throughout consecutive cell divisions that may result in various pathological phenotypes including abnormal mitotic events, such as unaligned metaphase, multipolar pre-anaphase, lagging chromosomes in anaphase, polar chromosomes (those located at the spindle poles) in metaphase or anaphase, chromosomal bridges in anaphase or telophase, mixed lagging chromosome and chromosomal bridges in anaphase, multipolar anaphase or telophase, or any combination thereof. Elevated chromosomal instability, resulting in abnormal mitotic events, has been correlated with poor prognosis in several cancers. Thus, the present application provides machine-learning models to analyze images of biological samples to characterize chromosomal instability in a patient sample (e.g., a tumor resection sample) using high throughput machine-learning models.

In one aspect, disclosed herein are methods and systems to train a machine-learning model with training histological images to identify individual mitotic events in input histological images and to output a chromosomal instability metric (e.g., one or more chromosomal instability metrics) to a user. In some embodiments, the output comprises an output of levels of different types or subtypes of chromosomal instability. Further, provided herein are methods and systems for characterizing a disease (e.g., cancer) in a patient from one or more histological images using a machine-learning model trained according to the methods of the present disclosure. The systems and methods can be applied, for example to a diagnostic or prognostic method, used to inform treatment selection (e.g., selection of a pharmaceutical drug), and/or evaluate the efficacy of a treatment, to further characterize a disease (e.g., cancer) or disease state (e.g., cancer state) in a patient.

Thus, in some aspects, provided herein is a method for characterizing a disease in a patient, comprising: inputting one or more input histological images of a biological sample into a trained machine-learning model trained using a plurality of annotated training histological images; identifying one or more mitotic events in the one or more input histological images using the trained machine-learning model, wherein the one or more mitotic events can be normal or abnormal mitotic events; determining a frequency of the abnormal mitotic events in the one or more input histological images based on the identified mitotic events; and classifying a pathological status of the one or more input histological images based on the determined frequency of abnormal mitotic events.

In some embodiments according to the method described above, the biological sample comprises at least a portion of a solid tumor. In some embodiments, at least a portion of the solid tumor is a biopsy slice of a solid tumor. In some embodiments, the biological sample relates to a plurality of training or input histological images from the same patient.

In some embodiments according to any of the methods described above, the one or more input histological images and/or the plurality of training histological images is an image captured at a resolution between 256 pixel×256 pixel and 10,000 pixel×10,000 pixel. In some embodiments, the one or more input histological images and/or the plurality of training histological images is captured between 20× and 100× magnification. In some embodiments, the one or more input histological images and/or the plurality of training histological images are hematoxylin and eosin (H&E) stained images.

In some embodiments according to any of the methods described above, the method further comprises segmenting one or more whole images into a plurality of image tiles, wherein the image tiles are inputted into the machine-learning model as the input histological images and/or the training histological images.

In some embodiments according to any of the methods described above, the machine-learning model segments the input histological images and/or the training histological images into tiles. In some embodiments, the input histological images and/or the training histological images are segmented by QuPath, U-Net, One Hundred Layers Tiramisu, or DenseNet. In some embodiments, the input histological images and/or the training histological images are processed to remove non-tumor tissue.

In some embodiments according to any of the methods described above, the annotated training histological images are annotated by an individual.

In some embodiments according to any of the methods described above, the annotated training histological images are annotated by a plurality of individuals. In some embodiments, the method further comprises selecting a set of high confidence annotated training histological images from the annotated training histological images. In some embodiments, the set of high confidence annotated training histological images are selected based on concordance between annotations performed by a plurality of individuals.

In some embodiments according to any of the methods described above, the annotated training histological images include negative instances, wherein the negative instances are training histological images without a mitotic event.

In some embodiments according to any of the methods described above, one or more of the input histological images and/or the plurality of training images are deposited into computer cloud storage.

In some embodiments according to any of the methods described above, the machine-learning model is an unsupervised model. In some embodiments, the machine-learning model is a weakly-supervised model. In some embodiments, the machine-learning model is a human-in-the-loop model. In some embodiments, the machine-learning model applies a model selected from the group consisting of Support Vector Machines (SVM), Random Forests (RF), Artificial Neural Network (ANN), Convolutional Neural Network (CNN), K-means, ResNet, DenseNet, eXtreme Gradient Boosting (XGBoost), VGG, swin-transformer, Faster-RCNN, Mask-RCNN, and CentralNet++.

In some embodiments according to any of the methods described above, the machine-learning model uses a probability metric for a predicted classification of the mitotic event. In some embodiments, the annotated events with confidence above a specified threshold are used to compute a chromosomal instability metric. In some embodiments, the chromosomal instability metric is the frequency of abnormal mitotic events with confidence above a specified threshold compared to all mitotic events in an input histological image. In some embodiments, the chromosomal instability metric is the frequency of abnormal mitotic events with confidence above a specified threshold compared to all mitotic events in an input histological image tile. In some embodiments, the chromosomal instability metric is the frequency of abnormal mitotic events with confidence above a specified threshold compared to all mitotic events in an input histological image tile normalized by a tumor area metric, wherein the tumor area metric is the tumor area calculated using a tumor image segmentor.

In some embodiments according to any of the methods described above, the machine-learning model outputs a chromosome instability metric for each input histological image tile. In some embodiments, the machine-learning model outputs a chromosome instability metric aggregated for all input histological image tiles. In some embodiments, the chromosomal instability metric is related to the pathological status of the input histological images. In some embodiments, the chromosomal instability metric is displayed to a user.

In some embodiments according to any of the methods described above, characterizing a disease comprises diagnosing the disease. In some embodiments, characterizing a disease comprises informing a treatment strategy. In some embodiments, characterizing a disease comprises evaluating the disease progression. In some embodiments, characterizing a disease comprises predicting the disease prognosis. In some embodiments, characterizing a disease comprises evaluating effect of a treatment. In some embodiments, characterizing a disease comprises identifying a patient population for treatment. In some embodiments, the disease is a cancer.

In some embodiments according to any of the methods described above, the method is implemented on a cloud-based computing platform.

In other aspects, provided herein is a system for characterizing a disease in a patient with machine-learning, comprising: one or more processors; a memory; and one or more programs with instructions for: receiving data representing one or more input histological images of a biological sample; identifying one or more mitotic events in the one or more input histological images using a trained machine-learning model trained using a plurality of annotated training histological images, wherein the one or more mitotic events can be normal or abnormal mitotic events; determining a frequency of the abnormal mitotic events in the one or more input histological images based on the identified mitotic events; and classifying a pathological status of the one or more input histological images based on the determined frequency of abnormal mitotic events.

In some embodiments according to the system described above, the biological sample comprises at least a portion of a solid tumor. In some embodiments, the at least a portion of the solid tumor is a biopsy slice of a solid tumor. In some embodiments, the biological sample relates to a plurality of training or input histological images from the same patient.

In some embodiments according to any of the systems described above, one or more input histological images and/or the plurality of training histological images is an image captured at a resolution between 256 pixel×256 pixel and 10,000 pixel×10,000 pixel. In some embodiments, one or more of the input histological images and/or the plurality of training histological images is captured at between 20× and 100× magnification. In some embodiments, one or more of the input histological images and/or the plurality of training histological images are hematoxylin and eosin (H&E) stained images.

In some embodiments according to any of the systems described above, the instructions further comprise instructions for segmenting one or more whole images into a plurality of image tiles, wherein the image tiles are inputted into the machine-learning model as the input histological images and/or the training histological images. In some embodiments, the machine-learning model segments the input histological images and/or the training histological images into tiles.

In some embodiments according to any of the systems described above, the annotated training histological images are annotated by an individual. In some embodiments, the input histological images and/or the training histological images are segmented by QuPath, U-Net, One Hundred Layers Tiramisu, or DenseNet. In some embodiments, the input histological images and/or the training histological images are processed to remove non-tumor tissue. In some embodiments, the annotated training histological images are annotated by an individual. In some embodiments, the annotated training histological images are annotated by a plurality of individuals. In some embodiments, a set of high confidence annotated training histological images are selected from the annotated training histological images. In some embodiments, the set of high confidence annotated training histological images are selected based on concordance between annotations performed by a plurality of individuals. In some embodiments, the annotated training histological images include negative instances, wherein the negative instances are training histological images without a mitotic event.

In some embodiments according to any of the systems described above, one or more of the input histological image and/or the plurality of training histological images are deposited into a computer cloud.

In some embodiments according to any of the systems described above, the machine-learning model is an unsupervised model. In some embodiments, the machine-learning model is a weakly-supervised model. In some embodiments, the machine-learning model is a human-in-the-loop model. In some embodiments, the machine-learning model applies a model selected from the group consisting of Support Vector Machines (SVM), Random Forests (RF), Artificial Neural Network (ANN), Convolutional Neural Network (CNN), K-means, ResNet, DenseNet, eXtreme Gradient Boosting (XGBoost), VGG, swin-transformer, Faster-RCNN, Mask-RCNN, and CentralNet++. In some embodiments, the machine-learning model uses a uses a probability metric for a predicted classification of the mitotic event and/or wherein the annotated events with confidence above a specified threshold are used to compute a chromosomal instability metric.

In some embodiments according to any of the systems described above, the machine-learning model uses a uses a probability metric for a predicted classification of the mitotic event. In some embodiments, the chromosomal instability metric is the frequency of abnormal mitotic events with confidence above a specified threshold compared to all mitotic events in an input histological image. In some embodiments, the chromosomal instability metric is the frequency of abnormal mitotic events with confidence above a specified threshold compared to all mitotic events in an input histological image tile. In some embodiments, the chromosomal instability metric is the frequency of abnormal mitotic events with confidence above a specified threshold compared to all mitotic events in an input histological image tile normalized by a tumor area metric. In some embodiments, the tumor area metric is the tumor area calculated using a tumor image segmentor.

In some embodiments according to any of the systems described above, the machine-learning model outputs a chromosome instability metric for each input histological image tile. In some embodiments, the machine-learning model outputs a chromosome instability metric aggregated for all input histological image tiles. In some embodiments, the chromosomal instability metric is related to the pathological status of the input histological image.

In some embodiments according to any of the systems described above, the chromosomal instability metric is displayed to a user.

In some embodiments according to any of the systems described above, characterizing a disease comprises diagnosing the disease. In some embodiments, characterizing a disease comprises informing a treatment strategy. In some embodiments, characterizing a disease comprises evaluating the disease progression. In some embodiments, characterizing a disease comprises predicting the disease prognosis. In some embodiments, characterizing a disease comprises evaluating effect of a treatment. In some embodiments, characterizing a disease comprises identifying a patient population for treatment. In some embodiments, the disease is a cancer.

In some embodiments according to any of the systems described above, the instructions are implemented on a cloud-based computing platform. In some embodiments, the instructions for implementing the instructions reside in cloud storage.

In other aspects, provided herein is a method for training a machine-learning model to analyze histological images of biological samples, comprising: annotating the plurality of training histological images by identifying both normal and abnormal mitotic events in the plurality of training histological images; and training the machine-learning model based on the annotated histological images, wherein the machine-learning model is configured to receive one or more input histological images and determine a pathological metric of the one or more input histological images.

In some embodiments according to a method for training described above, the biological sample comprises at least a portion of a solid tumor. In some embodiments, the at least a portion of the solid tumor is a biopsy slice of a tumor. In some embodiments, the biological sample relates to a plurality of training or input histological images from the same patient.

In some embodiments according to any of the methods for training described above, the one or more input histological images and/or the plurality of training histological images is an image captured at a resolution between 256 pixel×256 pixel and 10,000 pixel×10,000 pixel resolution. In some embodiments, the one or more input histological images and/or the plurality of training histological images is captured at between 20× and 100× magnification. In some embodiments, the one or more input histological images and/or the plurality of training histological images are hematoxylin and eosin (H&E) stained images.

In some embodiments according to any of the methods for training described above, the method further comprises segmenting one or more whole images into a plurality of image tiles, wherein the image tiles are inputted into the machine-learning model as the input histological images and/or the training histological images.

In some embodiments according to any of the methods for training described above, the machine-learning model segments the input histological images and/or the training histological images into tiles. In some embodiments, the input histological images and/or the training histological images are segmented by QuPath, U-Net, One Hundred Layers Tiramisu, or DenseNet. In some embodiments, the input histological images and/or the training histological images are processed to remove non-tumor tissue.

In some embodiments according to any of the methods for training described above, the annotated training histological images are annotated by an individual. In some embodiments, the individual is a person. In some embodiments, the person is a pathologist, a biologist, and/or a medical professional.

In some embodiments according to any of the methods for training described above, the annotating comprises identifying both normal and abnormal mitotic events in the training histological images. In some embodiments, the annotating comprises tracing the boundaries of both normal and abnormal mitotic events in the training histological images. In some embodiments, the annotating comprises classifying the mitotic events as normal or abnormal. In some embodiments, the annotating comprises classifying the normal and abnormal mitotic events with a level of confidence the mitotic event is normal or abnormal, respectively. In some embodiments, at least one of the abnormal mitotic event is selected from the group consisting of unaligned metaphase, multipolar pre-anaphase, lagging chromosomes in anaphase or telophase, chromosomal bridges in anaphase, mixed lagging chromosome and chromosomal bridges in anaphase or telophase, and multipolar anaphase. In some embodiments, at least one of the normal mitotic event is selected from the group consisting of prometaphase, normal metaphase, and normal anaphase. In some embodiments, the annotated training histological images are annotated by a plurality of individuals. In some embodiments, the method further comprises selecting a set of high confidence annotated training histological images from the annotated training histological images. In some embodiments, the set of high confidence annotated training histological images are selected based on concordance between annotations performed by a plurality of individuals. In some embodiments, the annotated training histological images include negative instances, wherein the negative instances are training histological images without a mitotic event.

In some embodiments according to any of the methods for training described above, one or more of the input histological image and/or the plurality of training histological images are deposited into computer cloud storage.

In some embodiments according to any of the methods for training described above, the machine-learning model is not trained using a genomic score.

In some embodiments according to any of the methods for training described above, the machine-learning model is an unsupervised model. In some embodiments, the machine-learning model is a weakly-supervised model. In some embodiments, the machine-learning model is a human-in-the-loop model. In some embodiments, the machine-learning model applies a model selected from the group consisting of Support Vector Machines (SVM), Random Forests (RF), Artificial Neural Network (ANN), Convolutional Neural Network (CNN), K-means, ResNet, DenseNet, eXtreme Gradient Boosting (XGBoost), VGG, swin-transformer, Faster-RCNN, Mask-RCNN, and CentralNet++.

In some embodiments according to any of the methods for training described above, the machine-learning model predicts a pathological metric for each input histological image. In some embodiments, the pathological metric is a chromosomal instability metric. In some embodiments, the chromosomal instability metric is related to the pathological status of the input histological image. In some embodiments, the chromosomal instability metric is the frequency of abnormal mitotic events compared to all mitotic events in an inputted histological image. In some embodiments, the chromosomal instability metric is the frequency of abnormal mitotic events compared to all mitotic events in an input histological image tile.

In some embodiments according to any of the methods for training described above, the machine-learning model outputs a chromosome instability metric for each input histological image tile. In some embodiments, the machine-learning model outputs a chromosome instability metric aggregated for all input histological image tiles.

In some embodiments according to any of the methods for training described above, the chromosomal instability metric is the number of abnormal mitotic events normalized by tumor size. In some embodiments, the chromosomal instability metric is the frequency of abnormal mitotic events with confidence above a specified threshold compared to all mitotic events in an input histological image tile normalized by a tumor area metric. In some embodiments, the tumor area metric is the tumor area calculated using a tumor image segmentor.

In some embodiments according to any of the methods for training described above, the chromosomal instability metric is displayed to a user.

In some embodiments according to any of the methods for training described above, the method is implemented on a cloud-based computing platform.

In other aspects, provided herein is a system for training a machine-learning model to analyze histological images of biological samples comprising one or more processors, memory, and one or more applications stored in the memory that include instructions for: receiving a plurality of annotated training histological images by identifying both normal and abnormal mitotic events in the plurality of training histological images; and training the machine-learning model based on the annotated histological images, wherein the machine-learning model is configured to receive one or more input histological images and determine a pathological metric of the one or more input histological images.

In some embodiments according to the system for training described above, the biological sample comprises at least a portion of a solid tumor. In some embodiments, the at least a portion of the solid tumor is a biopsy slice of a solid tumor. In some embodiments, the biological sample relates to a plurality of training or input histological images from the same patient.

In some embodiments according to any of the systems for training described above, the one or more input histological images and/or the plurality of histological images is an image captured at a resolution between 256 pixel×256 pixel and 10,000 pixel×10,000 pixel. In some embodiments, the one or more input histological images and/or the plurality of training histological images is captured at between 20× and 100× magnification. In some embodiments, the one or more input histological images and/or the plurality of training histological images are hematoxylin and eosin (H&E) stained images.

In some embodiments according to any of the systems for training described above, the instructions further comprise instructions for segmenting one or more whole images into a plurality of image tiles, wherein the image tiles are inputted into the machine-learning model as the input histological images and/or the training histological images.

In some embodiments according to any of the systems for training described above, the machine-learning model segments the input histological images and/or the training histological images into tiles. In some embodiments, the input histological images and/or the training histological images are segmented by QuPath, U-Net, One Hundred Layers Tiramisu, or DenseNet. In some embodiments, the input histological images and/or the training histological images are processed to remove non-tumor tissue.

In some embodiments according to any of the systems for training described above, the annotated training histological images are annotated by an individual. In some embodiments, the individual is a person. In some embodiments, the person is a pathologist, a biologist, and/or a medical professional. In some embodiments, the annotated training histological images are annotated by a plurality of individuals. In some embodiments, a set of high confidence annotated training histological images are selected from the annotated training histological images. In some embodiments, the set of high confidence annotated training histological images are selected based on concordance between annotations performed by a plurality of individuals. In some embodiments, the annotated training histological images include negative instances, wherein the negative instances are training histological images without a mitotic event. In some embodiments, the annotating comprises identifying both normal and abnormal mitotic events in the training histological images. In some embodiments, the annotating comprises tracing the boundaries of both normal and abnormal mitotic events in the training histological images. In some embodiments, the annotating comprises classifying the mitotic events as normal or abnormal. In some embodiments, the annotating comprises classifying the normal and abnormal mitotic events with a level of confidence the mitotic event is normal or abnormal, respectively. In some embodiments, at least one of the abnormal mitotic event is selected from the group consisting of unaligned metaphase, multipolar pre-anaphase, polar chromosomes in metaphase or anaphase, lagging chromosomes in anaphase or telophase, chromosomal bridges in anaphase or telophase, mixed lagging chromosome and chromosomal bridges in anaphase or telophase, and multipolar anaphase or telophase. In some embodiments, at least one of the normal mitotic event is selected from the group consisting of prometaphase, normal metaphase, normal anaphase, and normal telophase.

In some embodiments according to any of the systems for training described above, the one or more input histological images and/or the plurality of training histological images are deposited into computer cloud storage.

In some embodiments according to any of the systems for training described above, the machine-learning model is not trained using a genomic score.

In some embodiments according to any of the systems for training described above, the machine-learning model is an unsupervised model. In some embodiments, the machine-learning model is a weakly-supervised model. In some embodiments, the machine-learning model is a human-in-the-loop model. In some embodiments, the machine-learning model applies a model selected from the group consisting of Support Vector Machines (SVM), Random Forests (RF), Artificial Neural Network (ANN), Convolutional Neural Network (CNN), K-means, ResNet, DenseNet, eXtreme Gradient Boosting (XGBoost), VGG, swin-transformer, Faster-RCNN, Mask-RCNN, and CentralNet++.

In some embodiments according to any of the systems for training described above, the machine-learning model predicts a pathological metric for each input histological image. In some embodiments, the pathological metric is a chromosomal instability metric. In some embodiments, the chromosomal instability metric is related to the pathological status of the input histological image. In some embodiments, the chromosomal instability metric is the frequency of abnormal mitotic events compared to all mitotic events in an input histological image. In some embodiments, the chromosomal instability metric is the frequency of abnormal mitotic events compared to all mitotic events in an input histological image tile.

In some embodiments according to any of the systems for training described above, the machine-learning model outputs a chromosome instability metric for each input histological image tile. In some embodiments, the machine-learning model outputs a chromosome instability metric aggregated for all input histological image tiles. In some embodiments, the chromosomal instability metric is the number of abnormal mitotic events normalized by tumor size. In some embodiments, the chromosomal instability metric is the frequency of abnormal mitotic events with confidence above a specified threshold compared to all mitotic events in an input histological image tile normalized by a tumor area metric. In some embodiments, the tumor area metric is the tumor area calculated using an image segmentor.

In some embodiments according to any of the systems for training described above, the chromosomal instability metric is displayed to a user.

In some embodiments according to any of the systems for training described above, the instructions are implemented on a cloud-based computing platform. In some embodiments, the instructions for implementing the instructions reside in cloud storage.

In other aspects, provided herein is a non-transitory computer-readable storage medium storing one or more programs, the one or more programs comprising instructions, which when executed by one or more processors of an electronic device having a display, which when executed by an electric device cause the electronic device to: receive one or more input histological images of a biological sample; identify one or more mitotic events in the one or more input histological images using a trained machine-learning model trained using a plurality of annotated training histological images, wherein the one or more mitotic events can be normal or abnormal mitotic events; determine a frequency of the abnormal mitotic events in the one or more input histological images based on the identified mitotic events; and classify a pathological status of the one or more input histological images based on the determined frequency of abnormal mitotic events.

In some embodiments according to the computer-readable storage medium described above, the biological sample comprises at least a portion of a solid tumor. In some embodiments, the at least a portion of the solid tumor is a biopsy slice of a tumor. In some embodiments, the biological sample relates to a plurality of training or input histological images from the same patient.

In some embodiments according to any of the computer-readable storage mediums described above, the one or more input histological images and/or the plurality of training histological images is an images captured at a resolution between 256 pixel×256 pixel and 10,000 pixel×10,000 pixel. In some embodiments, the one or more input histological images and/or the plurality of training histological images is captured at between 20× and 100× magnification. In some embodiments, the one or more input histological images and/or the plurality training histological images are hematoxylin and eosin (H&E) stained images.

In some embodiments according to any of the computer-readable storage mediums described above, the instructions further comprise instructions for segmenting one or more whole images into a plurality of image tiles, wherein the image tiles are inputted into the machine-learning model as the input histological images and/or the training histological images.

In some embodiments according to any of the computer-readable storage mediums described above, the machine-learning model segments the input histological images and/or the training histological images into tiles. In some embodiments, the input histological images and/or the training histological images are segmented by QuPath, U-Net, One Hundred Layers Tiramisu, or DenseNet. In some embodiments, the input histological images and/or the training histological images are processed to remove non-tumor tissue.

In some embodiments according to any of the computer-readable storage mediums described above, the annotated training histological images are annotated by an individual. In some embodiments, the individual is a person. In some embodiments, the person is a pathologist, a biologist, and/or a medical professional. In some embodiments, the annotating comprises identifying both normal and abnormal mitotic events in the training histological images. In some embodiments, the annotating comprises tracing the boundaries of both normal and abnormal mitotic events in the training histological images. In some embodiments, the annotating comprises classifying the mitotic events as normal or abnormal. In some embodiments, the annotating comprises classifying the normal and abnormal mitotic events with a level of confidence the mitotic event is normal or abnormal, respectively. In some embodiments, at least one of the abnormal mitotic event is selected from the group consisting of unaligned metaphase, multipolar pre-anaphase, lagging chromosomes in anaphase, chromosomal bridges in anaphase, mixed lagging chromosome and chromosomal bridges in anaphase, and multipolar anaphase. In some embodiments, at least one of the normal mitotic event is selected from the group consisting of prometaphase, normal metaphase, and normal anaphase. In some embodiments, the annotated training histological images are annotated by a plurality of individuals. In some embodiments, the annotated training histological images are a set of high confidence annotated training histological images selected from the annotated training histological images. In some embodiments, the set of high confidence annotated training histological images are selected based on concordance between annotations performed by a plurality of individuals. In some embodiments, the annotated training histological images include negative instances, wherein the negative instances are training histological images without a mitotic event.

In some embodiments according to any of the computer-readable storage mediums described above, the machine-learning model is not trained using a genomic score.

In some embodiments according to any of the computer-readable storage mediums described above, the machine-learning model is an unsupervised model. In some embodiments, the machine-learning model is a weakly-supervised model. In some embodiments, the machine-learning model is a human-in-the-loop model. In some embodiments, the machine-learning model applies a model selected from the group consisting of Support Vector Machines (SVM), Random Forests (RF), Artificial Neural Net (ANN), Convolutional Neural Net (CNN), K-means, ResNet, DenseNet, eXtreme Gradient Boosting (XGBoost), VGG, swin-transformer, Faster-RCNN, Mask-RCNN, and CentralNet++.

In some embodiments according to any of the computer-readable storage mediums described above, the machine-learning model predicts a pathological metric for each input histological image. In some embodiments, the pathological metric is a chromosomal instability metric. In some embodiments, the chromosomal instability metric is related to the pathological status of the input histological image. In some embodiments, the machine-learning model uses a uses a probability metric for a predicted classification of the mitotic event. In some embodiments, the chromosomal instability metric is the frequency of abnormal mitotic events with confidence above a specified threshold compared to all mitotic events in an input histological image. In some embodiments, the chromosomal instability metric is the frequency of abnormal mitotic events with confidence above a specified threshold compared to all mitotic events in an input histological image tile.

In some embodiments according to any of the computer-readable storage mediums described above, the machine-learning model outputs a chromosome instability metric for each input histological image tile. In some embodiments, the machine-learning model outputs a chromosome instability metric aggregated for all input histological image tiles.

In some embodiments according to any of the computer-readable storage mediums described above, the chromosomal instability pathological metric is the number of abnormal mitotic events normalized by tumor size. In some embodiments, the chromosomal instability metric is the frequency of abnormal mitotic events with confidence above a specified threshold compared to all mitotic events in an input histological image tile normalized by a tumor area metric. In some embodiments, the tumor area metric is the tumor area calculated using a tumor image segmentor.

In some embodiments according to any of the computer-readable storage mediums described above, the chromosomal instability metric is displayed to a user.

In some embodiments according to any of the computer-readable storage mediums described above, the one or more computer programs are implemented on a cloud-based computing platform.

In some embodiments according to any of the computer-readable storage mediums described above, the instructions for implementing the one or more computer programs reside in cloud storage.

### BRIEF DESCRIPTION OF THE DRAWINGS

Various aspects of the disclosed methods and systems are set forth with particularity in the appended claims. A better understanding of the features and advantages of the disclosed methods and systems will be obtained by reference to the following detailed description of illustrative embodiments and the accompanying drawings, of which:

FIG. 1 illustrates a flowchart for training the chromosomal instability model according to examples of the disclosure.

FIG. 2 illustrates an exemplary process for tiling and annotating (e.g., with abnormal and normal mitotic events) a training histological image of a patient primary tumor, used to train the machine-learning model according to examples of the disclosure.

FIG. 3 illustrates a method for pre-training a chromosomal instability machine-learning model to diagnose a patient with a high chromosomal instability tumor, stratify patients based on a chromosomal instability metric, or recommend chromosomal instability specific treatments according to examples of the disclosure.

FIG. 4 is a histological image of an abnormal mitotic event according to an example of the disclosure.

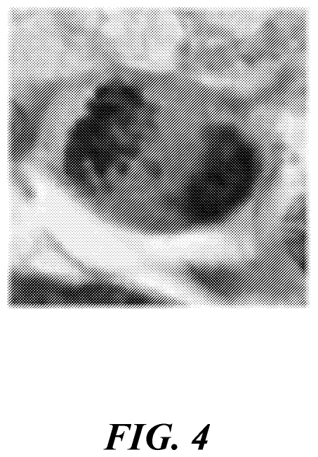

FIG. 5 illustrates a computing system in accordance with one or more examples of the present disclosure.

FIG. 6 illustrates a computing system comprising a cloud, in accordance with some embodiments.

FIG. 7 provides non-limiting examples of four histological images, and the corresponding labels annotating abnormal and normal mitotic events in those histological images, in accordance with some embodiments. In this example, annotated normal mitotic events include prometaphase and normal anaphase, and annotated abnormal mitotic events include unaligned metaphase, multipolar pre-anaphase, lagging chromosomes in anaphase, polar chromosomes (those located at the spindle poles) in metaphase or anaphase, chromosomal bridges in anaphase or telophase, mixed lagging chromosome and chromosomal bridges in anaphase or telophase, multipolar anaphase or telophase, or any combination thereof. Images are captured at 40× magnification and varying levels of resolution.

FIG. 8 illustrates the effect of a tumor segmentor method applied to a whole slide histological image.

FIG. 9 illustrates a concordance matrix comparing annotations performed by two experts.

FIG. 10. provides an exemplary custom DenseNet classifier designed to automatically classify normal and abnormal mitotic events.

FIG. 11. provides exemplary results for a 4-fold cross-validation experiment using methods disclosed herein.

FIG. 12 provides an exemplary CenterNet++ architecture for detection of anaphase figures.

FIG. 13 provides exemplary results for a CenterNet++method for detection of anaphase figures in a 4-fold cross-validation experiment.

FIG. 14 illustrates the true positive rate and false positive rate as a function of training samples.

FIG. 15 illustrates that performance improves with more negative samples. TPR and FPR is shown for various Reg-NS models using all 43K mitotic events. Each line represents a different ratio of negative to positives samples, from 0:1 to 1:1. Each star represents a different threshold from 0.15 to 0.60 with a step size of 0.05.

FIG. 16 provides a more detailed method for pre-training a chromosomal instability machine-learning model, expanding on the method provided in FIG. 3.

### DETAILED DESCRIPTION

The present application provides methods and systems to characterize chromosomal instability in a patient tumor sample that includes high throughput machine-learning models. Chromosomal instability describes the rate of abnormal cell division, mitosis, in a tumor sample. Because chromosomal instability has been linked to cancer stage, cancer prognosis, and probability of cancer treatment success, the disclosed methods and systems can help improve disease outcomes, and enable the discovery of new and novel therapeutic targets. Ultimately, these improvements may heighten the standard of care for patients with cancer.

Disclosed herein are methods and systems to train a machine-learning model with annotated training histological images (e.g., training histological images annotated with abnormal and normal mitotic events) to identify individual mitotic events in input histological images, and to output a chromosomal instability metric (e.g., a chromosomal instability score) to a user. The training histological images may be segmented into training histological image tiles. Each tile may be a segment of a whole training histological image. In some embodiments, the training histological images are segmented into a plurality of training histological image tiles prior to inputting the one or more training histological images into the machine-learning model. Specifically, the machine-learning model may be trained using annotated training histological images, whereby normal and abnormal mitotic events can be annotated in training the histological images. In some embodiments, the training histological images are annotated by an individual. In some embodiments, the individual is a human (e.g., a pathologist). In some embodiments, the training histological images are annotated by crowd sourcing.

In some embodiments, once the machine-learning model is trained, a tumor sample (e.g., a primary tumor sample from a patient) is prepared for input into the machine-learning model. In some embodiments, the tumor sample is prepared by an individual. In some embodiments, the tumor sample is prepared by a physician, pathologist, or other medical provider. The tumor sample may be a stained with hematoxylin and eosin (H&E) and fixed onto a slide for histological imaging. The histological images (e.g., “input histological images”) may be segmented into input histological image tiles. Each tile may be a segment of a whole input histological image. In some embodiments, the input histological images are segmented into a plurality of input histological image tiles prior to inputting the one or more input histological images into the machine-learning model. The segmenting may be implemented as part of the machine-learning model.

After segmenting the input histological image into input histological image tiles, the tiles of the histological image of the tumor sample can be inputted into the trained machine-learning. The machine-learning model may classify mitotic events (e.g., abnormal and normal mitotic events) in each input histological image tile, according to the normal and abnormal events that the model has been previously trained on. The results for each image tile can be aggregated to prepare a chromosomal instability metric based on a determined frequency of abnormal mitotic events, for the whole input histological image from the plurality of histological image tiles. The chromosomal instability metric may be used to characterize the pathological status of the one or more input histological images based on the determined frequency of abnormal mitotic events. Thus, in some embodiments, the chromosomal instability metric is used to classify the pathological status of the tumor sample from the patient.

In some embodiments, the present application provides a method for training a machine-learning model to analyze histological images of biological samples. In some embodiments the machine-learning model is trained with histological images of tumor samples. The histological image may be obtained from any source of histological images. In some embodiments, the training histological images of tumor samples are not publically available images (e.g., the training histological images are obtained from a private source, such as sourced form internal collaborations, licensed data, a clinic, a hospital system, a company or any other entity with access to histological images). In some embodiments, the training histological images of tumor samples are publically available images. The training histological images may be downloaded from a publically available database. In some embodiments, the publically available database may comprise images of one or more types of tumors. The training histological images, or a portion thereof, may be segmented into a plurality of training histological image tiles. In some embodiments, the training histological image tile is a segment of a whole training histological image. In some embodiments, the segmenting of the training histological image into training histological image tiles is conducted prior to training the machine-learning model. In some embodiments, the segmenting of the training histological image into training histological image tiles is implemented as part of training the machine-learning model.

In some embodiments, the training histological images or training histological image tiles are used to train a machine-learning model, such as any of the machine-learning models provided herein. In some embodiments, the machine-learning model is an unsupervised machine-learning model. An unsupervised machine-learning model may identify will identify and predict various classes of mitotic events (e.g., abnormal and normal mitotic events) with an associated probability aggregated by clustering methods, to determine a CIN metric. The clustering method may be any method described herein or known in the art. In some embodiments, the training histological images or training histological image tiles are used to train a weakly-supervised machine-learning model. In some embodiments, the training histological images or training histological image tiles are used to train a human-in-the-loop machine-learning model. The human-in-the-loop model may include training the machine-learning model with annotated training histological images.

The annotated training histological images or training histological image tiles may be annotated. In some embodiments, the training histological images or training histological image tiles are annotated by an individual. In some embodiments, the training histological images or training histological image tiles are annotated by more than one individual. In some embodiments, the individual is human. In some embodiments, the individual may be a pathologist, medical professional, biologist, or any other suitable individual capable of identifying mitotic events in a histological image. In some embodiments, crowd sourcing is used to annotate the training histological images or training histological image tiles thereof, e.g., annotated by an individual that does not perform the remainder of the machine-learning model training process and/or annotated by an individual that is not associated with the entity using the machine-learning model. The individual, such as any of the individuals described above, may identify normal and abnormal mitotic events in the training histological images. The abnormal mitotic events may include unaligned metaphase, multipolar pre-anaphase, lagging chromosomes in anaphase or telophase, chromosomal bridges in anaphase or telophase, mixed lagging chromosome and chromosomal bridges in anaphase or telophase, and multipolar anaphase or telophase. The normal mitotic events may include prometaphase, normal metaphase, and normal anaphase.

The machine-learning model may apply a Support Vector Machines (SVM), Random Forests (RF), Artificial Neural Network (ANN), Convolutional Neural Network (CNN), K-means, ResNet, DenseNet or eXtreme Gradient Boosting (XGBoost) model. The machine-learning model may apply an additional model known in the art or described herein.

Another aspect of the present application provides a method for characterizing a disease in a patient, using a machine-learning model configured to analyze histological images of biological samples.

In some embodiments, the biological sample can be any sample taken from the patient. In some embodiments, the biological sample from the patient comprise a slice of a biological tissues sample that has been biopsied for the disclosed methods or for other diagnostics, surveillance or treatment procedures. The biological tissue sample may be a biopsy of a solid tumor or a liquid tumor. One or more histology slides may be prepared for biological sample as described herein. The tissue may be stained with hematoxylin and eosin (H&E), or another tissue stain used in the art, as described herein. In some embodiments, the tissue is stained for centrosome amplification markers. In some embodiments, the tissue is stained using DAPI.

In some embodiments, the input histological image is an image captured of the prepared histology slide. The image may be captured at a resolution ranging from 256 pixel×256 pixel to 10,000 pixel×10,000 pixel. The resolution may depend on the magnification of the histological image. In some embodiments, the magnification is varied within a histological image or histological image tile to ensure the identification of mitotic events (e.g., abnormal and normal mitotic events). For example, some mitotic events require increased magnification in order to be visualized while other mitotic events require less magnification in order to be visualized. The image may be captured between about 20× and about 100× magnification. The input histological image may be a plurality of captured input histological image tiles from the same slide (e.g., the input histological image tiles are segments of a single whole input histological image). In some embodiments, the whole input histological image is segmented into a plurality of input histological image tiles prior to inputting the input histological image into the machine-learning model. In some embodiments, the whole input histological image is inputted into the machine-learning model, and the segmenting of the whole input histological image into a plurality of input histological image tiles is implemented as part of the machine-learning model. In some embodiments, the input histological image or plurality of input histological images, or input histological image tiles thereof, are deposited into computer cloud storage.

In some embodiments the input histological image or plurality of input histological image tiles are inputted into a trained machine-learning model that has been training using a plurality of annotated training histological images (e.g., annotated training histological image tiles). The machine-learning model may be trained to identify mitotic events in the input histological image or plurality of inputted histological image tiles. The machine-learning model may be trained to identify one or more normal and/or one or more abnormal mitotic events.

In some embodiments, the machine-learning model may be trained to output a chromosomal instability metric. The chromosomal instability metric may be calculated based on the identified mitotic events in the input histological image or the plurality of input histological image tiles.

In some embodiments, the invention provided herein can be used to benchmark the manner in which label acquisition and modeling choices interact to output a chromosome instability metric. Specifically, the methods look at trade-offs in the cost of label acquisition vs model performance by comparing the performance of classification models and object detection models. For label acquisition, classification models have lower costs, as they can be trained using comparatively easy-to acquire binary labels. Conversely, object detection models have higher costs in label acquisition, as they require detailed annotations. In some aspects, the impact on performance of different labeling schemes is also considered. For example, annotations from only one labeler can be compared to annotations from multiple labelers (per input). In other examples, the impact of using low-cost, noisy labels to high-cost, accurate labels can also be explored. Specifically, methods disclosed herein may use of expert labelers to annotate the histological images and/or non-expert crowdsourced labelers to annotate the histological images. In some aspects, the methods disclosed herein may use aggregated annotations (e.g., annotations from multiple individuals on the same histological image) or annotations from a single individual.

In some embodiments, the machine-learning model provided herein is used for object detection (e.g., detection of mitotic events, such as anaphase, in a histological image). In some embodiments, the machine-learning model detects mitotic events. In some embodiments, the machine-learning model detects anaphase figures. In some embodiments, the machine-learning model detects metaphase figures. In some embodiments, the machine-learning model provided herein is used for classification of mitotic events, such as anaphase (e.g., classification as abnormal anaphase or normal anaphase). In some embodiments, the machine-learning model detects anaphase figures in whole slide images. In some embodiments, \\the machine-learning model detects anaphase figures in tiled images. In some embodiments, the images (e.g., whole slide images or tiled images) have been stained, for example, using H&E staining, DAPI staining, or a stain identifying centrosome amplification markers. In some embodiments, the images are segmented. In some embodiments, the images are segmented to removed non-tumor area, e.g., by using a tumor segmentor method, such as any of the tumor segmentor methods described herein.

In some embodiments, the chromosomal instability metric is the frequency of abnormal mitotic events identified in an input histological image compared to all of the mitotic events identified in the input histological image. In some embodiments, the chromosomal instability metric is the frequency of abnormal mitotic events identified in an input histological image tile compared to all of the mitotic events identified in the input histological image tile. For example, if the machine-learning model identifies 2 abnormal mitotic events and 2 normal mitotic events, the chromosomal instability metric could be 0.5. In some embodiments, the chromosomal instability metric may be an aggregation of all the abnormal and normal mitotic events identified in all tiles belonging to the same whole input histological image. In some embodiments, the chromosomal instability metric may be an aggregation of all of the abnormal and normal mitotic events identified from one or more input histological images (or input histological image tiles thereof) belonging to the same tumor sample, from which the input histological image or images are captured. In some embodiments, the chromosomal instability metric may be an aggregation of all of the abnormal and normal mitotic events identified from one or more input histological images (or input histological image tiles thereof) belonging to the same patient. The chromosomal instability metric may be displayed to a user.

In some embodiments, the machine-learning model uses high quality mitotic events for the chromosomal instability metric. The machine-learning model may use a probability metric for a predicted classification of the mitotic event. The annotated events with a score above a specified threshold may be used to compute the chromosomal instability metric. For example, the specified threshold is used to filter and aggregate the frequency of the abnormal mitotic events to compute the chromosomal instability metric.

In some embodiments, the chromosomal instability metric relates to the pathological status of the inputted histological image. The pathological status of the inputted histological image may be used to characterize the disease for the patient. The pathological status may be used to diagnose a patient, inform a treatment strategy, evaluate disease progression, determine disease state, predict the disease prognosis, evaluate effect of a treatment, identify if the patient is part of a larger patient population, or for any other application in the art. For example, if a patient's biological sample has a high chromosomal instability metric corresponding to a high frequency of abnormal mitotic events, a physician may prescribe an anti-chromosomal instability therapeutic as described herein.

### I. Definitions

Although the following description uses terms “first.” “second,” etc. to describe various elements, these elements should not be limited by the terms. These terms are only used to distinguish one element from another. For example, a first graphical representation could be termed a second graphical representation, and, similarly, a second graphical representation could be termed a first graphical representation, without departing from the scope of the various described embodiments. The first graphical representation and the second graphical representation are both graphical representations, but they are not the same graphical representation.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including.” “comprises.” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if”′ is, optionally, construed to mean “when” or “upon” or “in response to determining” or “in response to detecting.” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

The term “chromosomal instability”, “chromosome instability”, or “CIN” refers to chromosome segregation errors throughout consecutive mitotic events and comprises both structural and numerical CIN, as used in the art.

As used herein, the term “annotate”, “annotated”, or “annotating” refers to adding extra information. An image, such as a training histological image or training histological image tiles is annotated to include extra information regarding mitotic events (e.g., annotated to identify and classify mitotic events). Annotating comprises adding text to an image, highlighting a portion of an image, including numerical information on an image, color-coding a portion of an image, and drawing a shape around a portion of an image. Annotating may be implemented by any skilled person in the art.

The term “abnormal mitotic events” refers to mitotic events wherein the chromosomes do not correctly match and align at the mitotic plate and/or do not segregate correctly into the resulting daughter cells. Abnormal mitotic events comprise unaligned metaphase, multipolar pre-anaphase, polar chromosomes in metaphase or anaphase, lagging chromosomes in anaphase, chromosomal bridges in anaphase (e.g., bridged-anaphase) or telophase, mixed lagging chromosome and chromosomal bridges in anaphase (e.g., mixed-anaphase) or telophase, and multipolar anaphase or telophase. Abnormal mitotic events also include situations where chromosomes segregate into 3 or more daughter cells instead of the standard 2 daughter cells.

The term “normal mitotic events” refers to mitotic events wherein the chromosomes correctly match and align at the mitotic plate and segregate with one chromatid of each chromosome segregating into each of the two resulting daughter cells. Normal mitotic events may refer to mitotic events that result in normal cellular development and growth, cellular replacement, repair, and regeneration. Normal mitotic events may include mitotic events with slight segregation errors. Normal mitotic events comprise prometaphase, normal metaphase, normal anaphase, and normal telophase.

The term “image feature” refers to a property of an image or image patch that contains information about the content of the image or image patch, e.g., image features may be specific structures in the image or image patch such as points, edges, shapes, textures, or objects, or may be non-visual or non-human-interpretable properties of the image derived from an image processing- and/or machine-learning-based analysis of an image.

As used herein, the terms “classification model” and “classifier” are used interchangeably, and refer to a machine-learning architecture or model that has been trained to sort input data into one or more labeled classes or categories.

The terms “treat,” “treating.” and “treatment” are used synonymously herein to refer to any action providing a benefit to a subject afflicted with a disease state or condition, including improvement in the condition through lessening, inhibition, suppression, or elimination of at least one symptom, delay in progression of the disease or condition, delay in recurrence of the disease or condition, or inhibition of the disease or condition. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. In reference to a cancer, the number of cancer cells present in a subject may decrease in number and/or size and/or the growth rate of the cancer cells may slow. In some embodiments, treatment may prevent or delay recurrence of the disease. In the case of cancer, the treatment may: (i) reduce the number of cancer cells; (ii) inhibit, retard, slow to some extent and preferably stop cancer cell proliferation; (iii) prevent or delay occurrence and/or recurrence of the cancer; and/or (iv) relieve to some extent one or more of the symptoms associated with the cancer. The methods of the invention contemplate any one or more of these aspects of treatment.

As used herein, the term “cloud” refers to shared or sharable storage of software and/or electronic data using, e.g., a distributed network of computer servers. In some instances, the cloud may be used, e.g., for archiving electronic data, sharing electronic data, and analyzing electronic data using one or more software packages residing locally or in the cloud.

The section headings used herein are for organizational purposes only and are not to be construed as limiting the subject matter described.

Features and preferences described above in relation to “embodiments” are distinct preferences and are not limited only to that particular embodiment; they may be freely combined with features from other embodiments, where technically feasible, and may form preferred combinations of features. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

### II. Samples and Sample Preparation

The methods disclosed herein may be used to analyze a diverse array of samples (e.g., biological samples). In some embodiments, the biological sample comprises at least a portion of a solid tumor or a liquid tumor. The biological sample may be stained, and subsequently imaged, for use in the methods provided herein.

A sample disclosed herein can be or can be derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In some embodiments, the sample includes a tumor sample.

Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy for said disease. In some embodiments, the biological sample is from an individual subject. In some embodiments, the individual subject is a mammal, such as a human, bovine, horse, feline, canine, rodent, or primate. In some embodiments, the biological sample is a mouse sample. The mouse may be a mouse tumor model or a human cancer mouse xenograft model. In some embodiments, the method comprises obtaining the biological sample from an individual (e.g., human). The biological sample can be obtained as a tissue sample, such as a tissue section, biopsy, a core biopsy, needle aspirate, or fine needle aspirate. In some embodiments, the sample can be a fluid sample, such as a blood sample, urine sample, or saliva sample. In some embodiments, the sample can be a skin sample, a colon sample, a check swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface (e.g., a substrate), such as a glass slide.

Biological samples can include one or more diseased cells. A diseased cell can have altered metabolic properties, gene expression, protein expression, and/or morphologic features, such as altered chromosome morphology and/or altered mitotic state. Examples of diseases include inflammatory disorders, metabolic disorders, nervous system disorders, and cancer.

In some embodiments, the cancer is a solid tumor or a hematologic malignancy (e.g., a liquid tumor). In some embodiments, the cancer is a carcinoma, a sarcoma, a lymphoma, or a leukemia. In some instances, the cancer is a naive cancer, or a cancer that has not been treated by a particular therapeutic agent. In some embodiments, the cancer has previously been treated by a particular therapeutic agent. In some embodiments, the cancer is a primary tumor or a primary cancer, which is a tumor that originated in the location or organ in which it is present and did not metastasize to that location from another location. In some embodiments, the cancer is a metastatic cancer. In some embodiments, the cancer is a relapsed or refractory cancer.

In some embodiments, a tumor or cancer originates from blood, lymph node, liver, brain/neuroblastoma, esophagus, trachea, stomach, intestine, colon, rectum, anus, pancreas, throat, tongue, bone, ovary, uterus, cervix, peritoneum, prostate, testes, breast, kidney, lung, or skin, gastric, colorectal, bladder, head and neck, nasopharyngeal, endometrial, bile duct, oral, multiple myeloma, leukemia, soft tissue sarcoma, gall bladder, endocrine, mesothelioma, wilms tumor, duodenum, neuroendocrine, salivary gland, larynx, choriocarcinoma, cardial, small bowel, eye, or germ cell cancer.

In some embodiments, a cancer (e.g., a primary tumor) includes, but is not limited to, acute lymphoblastic leukemia (ALL), acute myeloid leukemia (AML), bladder cancer, breast cancer, brain cancer, cervical cancer, colon cancer, colorectal cancer, endometrial cancer, gastrointestinal cancer, glioma, glioblastoma, head and neck cancer, kidney cancer, liver cancer, lung cancer, lymphoid neoplasia, melanoma, a myeloid neoplasia, ovarian cancer, pancreatic cancer, prostate cancer, squamous cell carcinoma, testicular cancer, stomach cancer, or thyroid cancer. In some instances, a cancer includes a lymphoid neoplasia, head and neck cancer, pancreatic cancer, endometrial cancer, colon or colorectal cancer, prostate cancer, glioma or other brain/spinal cancers, ovarian cancer, lung cancer, bladder cancer, melanoma, breast cancer, a myeloid neoplasia, testicular cancer, stomach cancer, cervical, kidney, liver, or thyroid cancer.

In some embodiments, cancer cells can be derived from solid tumors, hematological malignancies, cell lines, or obtained as circulating tumor cells. Biological samples can also include fetal cells and immune cells. In some embodiments, the biological sample comprises at least a portion of a tumor sample. In some embodiments, the biological sample comprises cancer cells derived from liquid tumors. In some embodiments, the biological sample comprises cancer cells derived from solid tumors. In some embodiments, the biological sample comprises a solid tumor, or portion thereof, and is obtained from a tissue biopsy from an individual suspected of having a disease (e.g., cancer).

In some embodiments, a substrate herein can be any support that is insoluble in aqueous liquid and which allows for positioning of biological samples on the support. In some embodiments, the biological sample can be attached to a substrate. In some embodiments, the substrate facilitates the visualization of the morphology of the biological sample. In some embodiments, the sample can be attached to the substrate reversibly by applying a suitable polymer coating to the substrate, and contacting the sample to the polymer coating. In some embodiments, the sample can be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A biological sample can be harvested from a subject (e.g., via surgical biopsy or whole subject sectioning) or grown in vitro as a population of cells, and prepared for analysis as a tissue slice or tissue section. In some embodiments, the samples grown in vitro may be sufficiently thin for analysis without further processing steps. Alternatively, the samples grown in vitro, and samples obtained via biopsy or sectioning, can be prepared as thin tissue sections using a mechanical cutting apparatus such as a vibrating blade microtome. In some embodiments, a thin tissue section can be prepared by applying a touch imprint of a biological sample to a suitable substrate material.

The thickness of a tissue section typically depends on the method used to prepare the section and the physical characteristics of the tissue, and therefore sections having a wide variety of different thicknesses can be prepared and used for the methods described herein. For example, in some embodiments, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. In some embodiments, the thickness of the tissue section can be greater than about 100, 90, 80, 70, 60, 50, 40, 30, 20, 15, 14, 13, 12, 11, 10, 9, 8, 7, 6, 5, 4, 3, 2, 1.5, 1.0, 0.7, 0.5, 0.4, 0.3, 0.2, 0.1 μm or less. In some embodiments, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, however, sections with thicknesses larger or smaller than these ranges can also be analyzed using the machine-learning model described herein.

Multiple sections can also be obtained from a single biological sample. For example, in some embodiments, multiple tissue sections can be obtained from a surgical biopsy sample by performing serial sectioning of the biopsy sample using a sectioning blade. In some embodiments, spatial information of the biopsy sample is maintained through the serial sectioning process.

In some embodiments, the biological sample (e.g., a tissue section from a tumor biopsy, as described above) can be prepared by deep freezing at a temperature suitable to maintain or preserve the integrity (e.g., the physical characteristics) of the tissue structure. In some embodiments, the frozen tissue sample can be sectioned, such as thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. In some embodiments, the tissue sample is prepared at a temperature that is, for example, less than −15° C., less than −20° C., or less than −25° C.

In some embodiments, the biological sample can be prepared using formalin-fixation and paraffin-embedding (FFPE). In some embodiments, cell suspensions and other non-tissue samples can be prepared using formalin-fixation and paraffin-embedding. In some embodiments, following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples.

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample comprises cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347 (6221): 543-548, 2015, the entire contents of which are incorporated herein by reference.

To facilitate visualization, biological samples can be stained using a wide variety of stains and staining techniques. In some embodiments, the visualization comprises visualization of micronuclei, chromatin, and/or mitotic events. In some embodiments, the visualization comprises visualization of mitotic events. In some embodiments, the mitotic events may be normal and/or abnormal mitotic events.

In some embodiments, for example, a biological sample can be stained using any number of stains, including but not limited to, Feulgen, acridine orange, DAPI, eosin, ethidium bromide, and haematoxylin. In some embodiments, the same is stained for centrosome amplification markers. In some embodiments, the sample is stained using hematoxylin and eosin (H&E) staining techniques. In some embodiments, the stained biological samples are imaged. Image processing techniques are described in Section III.A.

### III. Methods

The methods provided herein comprise methods for training a machine-learning model, and methods of characterizing a disease in a patient using a machine-learning model trained using the training methods described herein. In some embodiments, the methods include training a machine-learning model using a plurality of annotated training histological images, or image tiles thereof. In some embodiments, the methods include characterizing a disease in a patient using input histological images, or image tiles thereof.

### A. Image Processing

The disclosed methods (e.g., methods of training a machine-learning model and/or methods of charactering a disease in a patient using a machine-learning model trained using the method of training a machine-learning model described herein) may be utilized with images, e.g., whole slide pathology images or tiles thereof, of tissue samples that have been acquired using any of a variety of microscopy imaging techniques known to those of skill in the art. Examples of imaging techniques include, but are not limited to, bright-field microscopy, dark-field microscopy, phase contrast microscopy, differential interference contrast (DIC) microscopy, fluorescence microscopy, confocal microscopy, confocal laser microscopy, super-resolution optical microscopy, scanning or transmission electron microscopy, and the like.

The imaging techniques described herein may be used to capture training images and/or input images. In some embodiments, the training images and/or input images are histological images, such as training histological images and input histological images. In some embodiments, the imaging techniques are used to capture stained (e.g., hematoxylin and eosin (H&E) stained) training histological images of a biological sample. In some embodiments, the imaging techniques are used to capture stained (e.g., H&E stained) input histological images of a biological sample. In some embodiments, the biological sample comprises a tumor sample, such as at least a portion of a solid tumor or a liquid tumor (e.g., a biopsy slice of a solid tumor). In some embodiments, the biological sample comprises any of the biological samples described in Section II.

In some embodiments, the plurality of training histological images are segmented into tiles prior to imaging. In some embodiments, the plurality of training histological images are segmented into tiles after imaging. In some embodiments, the input histological image is segmented into tiles prior to imaging. In some embodiments, the input histological image is segmented into tiles after imaging. In some embodiments, the imaging is implemented at part of a machine-learning model, such as any of the machine-learning models described herein.

In some embodiments, microscopy is used to capture a digital image of the biological sample (e.g., a biological sample comprising a tumor sample). In some embodiments, the biological sample is stained (e.g., H&E stained) prior to microscopy imaging. In some aspects, bright-field microscopy is used to capture histological images of the biological sample. In bright-field microscopy, light is transmitted through the biological sample and the contrast is generated by the absorption of light in dense areas of the biological sample. In some embodiments, the biological sample is stained (e.g., H&E stained) prior to bright-field imaging.

In some aspects, fluorescence microscopy is used to capture a digital image of the biological sample (e.g., a biological sample comprising a tumor sample). In some embodiments, the biological sample is stained (e.g., H&E stained) prior to fluorescence imaging. In some aspects, a fluorescence microscope is an optical microscope that uses fluorescence and phosphorescence instead of, or in addition to, reflection and absorption, to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter.

In some embodiments, the plurality of training histological images, or tiles thereof, and/or input histological images, or tiles thereof, are captured digitally. In some embodiments, digital pathology slide scanners are used to capture images of biological samples. In some aspects, the digital pathology slide scanners can capture bright-field or fluorescent images. In some aspects, the digital pathology slide scanners have automated stages to automate imaging of a whole slide. The digital pathology slide scanners may also allow for Z stack images and/or live viewing of the biological sample. In some embodiments, the digital pathology slide scanners may hold around 1, 5, 10, 20, 30, 40, 50, 60, 70, 80, 90, 100 or more individual slides of biological samples. In some aspects, the digital pathology slide scanners may hold around 100, 150, 200, 250, 300, 350, 400, 450, 500, or more individual slides of biological samples. In some aspects, the digital histological images (e.g., digital histological training images, digital histological input images, or tiles thereof) are automatically uploaded to a computer or mobile device.

In some embodiments, histological images (e.g., training histological images, or tiles thereof, and/or input histological images, or tiles thereof) of the biological sample are captured with magnification. In some embodiments, the histological images are captured at about 20×, 25×, 30×, 35×, 40×, 45×, 50×, 55×, 60×, 65×, 70×, 75×, 80×, 90×, 95×, 100× or more magnification. In some embodiments, the magnification may be about 20× or greater, 25× or greater, 30× or greater, 35× or greater, 40× or greater, 45× or greater, 50× or greater, 55× or greater, 60× or greater, 65× or greater, 70× or greater, 75× or greater, 80× or greater, 85× or greater, 90× or greater, or 95× or greater. In some embodiments, the magnification may be about 100× or less, 95× or less, 90× or less, 85× or less, 80× or less, 75× or less, 70× or less, 65× or less, 60× or less, 55× or less, 50× or less, 45× or less, 40× or less, 35× or less, 30× or less, 25× or less, 20× or less. In some embodiments, the image magnification may be between about 20× and about 100×, such as between any of about 20× to about 50×, about 35× to about 75×, or about 50× to about 100×. In some embodiments, the magnification varies across images tiles of a segmented whole slide histological image.

The magnification of the plurality of training histological images, or tiles thereof, and/or input histological images, or tiles thereof, may be achieved through eyepiece lenses, objective lenses, or a combination of thereof.

In some aspects, histological images (e.g., training histological images, or tiles thereof, and/or input histological images, or tiles thereof) are captured at high resolutions. In some embodiments, the high resolution capture of the histological images allows for detection of individual cells within a tissue. In some embodiments, the high resolution capture of the histological images allows for detection individual mitotic events (e.g., abnormal or normal mitotic events) within a cell within a tissue. In some embodiments, the resolution varies across a whole slide histological image. In some embodiments, the resolution varies across image tiles of a segmented whole slide histological image. High resolution histological images or histological image tiles may be captured a resolution between about 256×256 pixels to about 10,000×10,000 pixels. In some embodiments, high resolution histological images or histological image tiles may be about 256×256 pixels, 300×300 pixels, 400×400 pixels, 500×500 pixels, 600×600 pixels, 700×700 pixels, 800×800 pixels, 900×900 pixels, 1,000×1,000 pixels, 2,000×2,000 pixels, 3,000×3,000 pixels, 4,000×4,000 pixels, 5,000×5,000 pixels, 6,000×6,000 pixels, 7,000×7,000 pixels, 8,000×8,000 pixels, 9,000×9,000 pixels, 10,000×10,000 pixels, or more. In some embodiments, the resolution of the histological image or histological image tile may include the resolutions and ranges above those described. In some embodiments, the resolution of the histological image or histological image tile may depend on the magnification of the histological image.

The disclosed methods may be utilized with histological images (e.g., training histological images, or tiles thereof, and/or input histological images, or tiles thereof), e.g., whole slide pathology (histological) images, of tissue samples that have been acquired using any of a variety of microscopy imaging techniques known to those of skill in the art. Examples include, but are not limited to, bright-field microscopy, dark-field microscopy, phase contrast microscopy, differential interference contrast (DIC) microscopy, fluorescence microscopy, confocal microscopy, confocal laser microscopy, super-resolution optical microscopy, scanning or transmission electron microscopy, and the like.

Any of a variety of image processing methods known to those of skill in the art may be used for image processing/pre-processing of the histological images (e.g., training histological images, or tiles thereof, and/or input histological images, or tiles thereof) described herein. Examples include, but are not limited to, Canny edge detection methods, Canny-Deriche edge detection methods, first-order gradient edge detection methods (e.g., the Sobel operator), second order differential edge detection methods, phase congruency (phase coherence) edge detection methods, other image segmentation methods (e.g., intensity thresholding, intensity clustering methods, intensity histogram-based methods, etc.), feature and pattern recognition methods (e.g., the generalized Hough transform for detecting arbitrary shapes, the circular Hough transform, etc.), and mathematical analysis methods (e.g., Fourier transform, fast Fourier transform, wavelet analysis, auto-correlation, etc.), or any combination thereof.

In some embodiments, the methods provided herein further comprise segmenting one or more of the input or training histological images into a plurality of histological image tiles prior to inputting the one or more input or training histological images into the machine-learning model. In some embodiments, the segmenting is implemented as part of the machine-learning model. In some embodiments, the whole histological images (e.g., whole training histological images and/or whole input histological images) are segmented into smaller image tiles. In some embodiments, the image tile is a segment of a whole histological image. In some embodiments, at least a portion of the plurality of training histological images is a tile of a whole training histological image. In some embodiments, at least a portion of the one or more whole input histological images is a tile of a whole input histological image.

In some embodiments, the image tiles are extracted from a whole histological image (e.g., whole training histological image and/or whole input histological image) using masking or other image processing techniques such as those described above. In some embodiments, a sliding window approach can be used to extract tiles from a whole histological image. In some aspects, about 5, 10, 15, 20, 25, 30, 40, 50, 60, 70, 80, 90, 100, 200, 400, 600, 800, 1,000, 1,500, 2,000, 2,500, 3,000, 3,500, 4,000, 4,500, 5,000, or more than 5,000 image tiles, may be extracted from each whole histological image of a plurality of whole histological images. In some instances, the number of image tiles (e.g., image patches) extracted from each whole histological image may have any value within the range of values described in this paragraph, e.g., 1,224 image tiles.

In some embodiments, the image tiles (e.g., images patches) extracted from a whole histological image (e.g., whole training histological image and/or whole input histological image) may be of different sizes. In some embodiments, the image tiles extracted from a histological image may all be of the same size. In some embodiments, the image tiles extracted from a whole histological image may be of a predetermined size. In some embodiments, the image tiles may all be of the same size when training a machine-learning model for a particular classification application, in order to ensure that the image tile patterns processed by the machine-learning model are consistent in terms of, e.g., field of view, and to ensure that the machine-learning model's weights and the patterns learned are meaningful. In some instances, image tile size may be varied from experiment to experiment or from application to application. In some instances, image tile size can be considered a tunable parameter during the training method, such as any of the methods of training a machine-learning described herein.

In some embodiments, the image tile size (or a range of image tile sizes) is determined by the input histological image size expected to be characterized by the trained machine-learning model, or by other image size considerations. In some aspects, the size of the image tile may range from about 10 pixels to about 200 pixels. In some aspects, the size of the image tile may be at least 10 pixels, at least 50 pixels, at least 75 pixels, at least 100 pixels, at least 105 pixels, more. In some aspects, the size of the image tile may be at most 500 pixels, at most 200 pixels, at most 150 pixels, at most 120 pixels, at most 100 pixels, or at most 10 pixels. Any of the lower and upper values described in this paragraph may be combined to form a range included within the present disclosure, for example, in some instances the size of the image tile may range from about 100 pixels to about 105 pixels. Those of skill in the art will recognize that the size of the image tile may have any value within this range, e.g., about 2.8×103 pixels.

In some embodiments, the image tiles may be of a square or rectangular shape, e.g., 100 pixels×100 pixels, or 1,000 pixels×1,000 pixels. In some instances, the image tiles may be of irregular shape. In some embodiments, the histological images or the histological image tiles may be extracted from 1, 2, 3, 4, 5, 6, 7, 8, 9, 10 or more than 10 histological images of the same biological tissue.

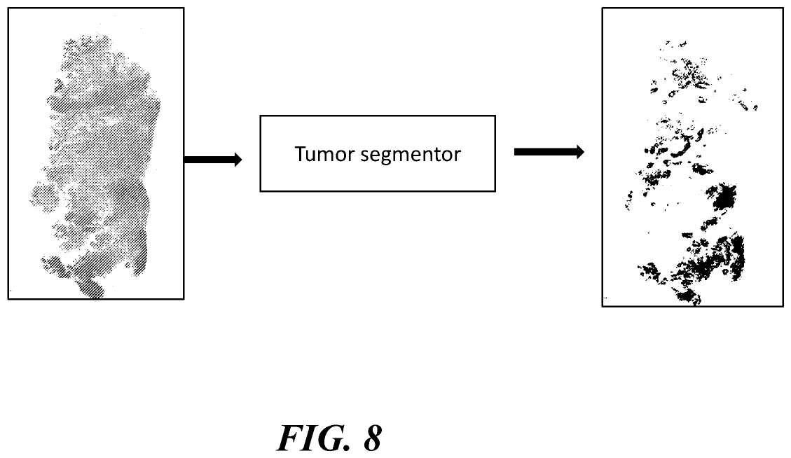

In some embodiments, tumor segmentor methods are used to process the images (e.g., whole slide images) or image tiles. In some embodiments, the tumor segmentor method removes non-tumor area from the whole slide image or image tile. In some embodiments, the tumor segmentor method removes all of the non-tumor area from the whole slide image or image tile. In some embodiments, the tumor segmentor method partially removes the non-tumor area from the whole slide image or image tile. The tumor segmentor method may make future anaphase detection more efficient by removing non-tumor areas, such as stroma and fat, from the image, as shown in FIG. 8. The tumor segmentor method may be semi-automatic segmentation with QuPath in whole slide images. In some embodiments, an expert may rely on their knowledge to segment the image. In some embodiments, U-Net, QuPath, One Hundred Layers Tiramisu, or DenseNet segmentation methods can be used to identify tumor tissue in whole slide images or image tiles. The tumor segmentor method may also output the area of tumor tissue in the whole slide image or image tile, which can be used to standardize the measured CIN from WSI.

In some embodiments, the tumor segmentor method reduces the area of the whole slide image or image tile. In some embodiments, the tumor segmentor method reduces the search area for the machine-learning model. In some embodiments, the reduced search area comprises tumor tissue. In some embodiments, after applying the tumor segmentor method, the search area for the machine-learning model is reduced compared to removing only white background from the whole slide image or image tile. In some embodiments, after applying the tumor segmentor method, the search area for the machine-learning model is reduced by about 5% or greater compared to the whole slide image or image tile, such as reduced by about 10%, 20%, 30%, 40%, 50%, or greater compared to the whole slide image or image tile.

### B. Training Data

The type of training data used for training a machine-learning model for use in the disclosed methods of characterizing a disease in a patient will depend on, for example, whether a supervised or unsupervised approach is taken, as well as on the objective to be achieved. In some instances, the training data may be continuously updated and used to update the machine-learning model(s) in a local or distributed network of one or more deployed pathology (histological) image analysis systems in real time. In some cases, the training data may be stored in a training database that resides on a local computer or server. In some cases, the training data may be stored in a training database that resides online or in a cloud.

In some embodiments, the machine-learning model is trained using a plurality of training histological images. In some embodiments, the training histological images comprise an image of a biological sample. In some embodiments, the biological sample comprises a tumor sample (e.g., a tumor sample or a portion thereof, obtained from a tumor slice of a patient). In some embodiments, the training histological images are stained images. In some embodiments, the training histological images are processed and captured according to any of the techniques described herein.

In some aspects, the training histological images may obtained from any source. In some embodiments, the machine-learning model is trained with histological images that are not publically available. In some embodiments, the training histological images are obtained from a private source, such as an internal collaboration or licensed data. The training histological images may come from a clinic, a hospital system, a company or any other entity with access to histological images. In some embodiments, the training histological images are publically available images. The training histological images may be downloaded from a publically available database. In some aspects, the training histological images may be obtained from a publically available database. The publically available database may be a pan-cancer database. The publically available databases may be available from, The Cancer Genome Atlas (TCGA) Pan-Cancer Atlas, Clinical Proteomic Tumor Analysis Consortium (CPTAC), National Lung Screening Trial, Osteosacoma Pathology, Prostate Fused-MRI-Pathology, Prostate-MRI, Osteosarcoma Tumor Assessment, Lung Fused-CT-Pathology, AML-Cyuomorphology_LMU, Post-NAT-BRCA, SNL-Breast, C-NMC 2019, MiMM_SBILab, SN-AM, IvyGAP, or any other database with a histological image dataset. TCGA is a cancer genomics program containing over 20,000 primary cancer and matched normal samples corresponding to 33 cancer types. The data is publicly available. Breast cancer is one of the most common types of cancer and correspondingly is one of the largest sample sizes on TCGA (TCGA BRCA, 1098 cases) (The Cancer Genome Network, 2012).

In some embodiments, the training histological images are obtained from a mouse. In some embodiments, the mouse is a mouse tumor model. In some embodiments, the mouse is a human cancer mouse xenograft model. Training histological images obtained from mice may be advantageous due to a high instance of mitotic event (e.g., a higher instance of mitotic events compared to a training histological image obtained from a human). In some embodiments, the mouse training histological image has more total mitotic events compared to a human training histological image. In some embodiments, the mouse training histological image has more abnormal mitotic events compared to a human training histological image.

In some embodiments, the machine-learning model is trained with histological images that are obtained from more than one source. For example, the machine-learning model can be trained with human histological images (e.g., obtained from TCGA) and mouse histological images. The source for the training histological images may be selected based on which histological images have sufficient training data for the machine-learning model, such as which histological images have sufficient mitotic events and/or sufficient abnormal mitotic events. In some embodiments, the machine-learning model is trained with histological images that come from 2, 3, 4, 5, 6, 7, 8, 9, 10 or more image datasets. In some embodiments, the machine-learning model is trained with a subset of histological images from one source.

In some embodiments, the machine-learning model is trained with about 1,000 histological images or image tiles thereof. In some embodiments, the machine-learning model is trained with about 100, 500, 1,000, 2,000, 3,000, 4,000, 5,000, 6,000, 7,000, 8,000, 9,000, 10,000, or more, histological images or image tiles thereof.

In some embodiments, the machine-learning model is trained with training histological images or tiles of training histological images captured from about 1,000 biological samples. In some embodiments, the biological samples are biological samples from the same patient. In some embodiments, at least a portion of the biological samples are biological samples from different patients. In some embodiments, the biological sample is a biological sample from a different patient. In some embodiments, the machine-learning model is trained with training histological images or training histological image tiles from about 100, 500, 1,000, 2,000, 3,000, 4,000, 5,000, 6,000, 7,000, 8,000, 9,000, 10,000, or more biological samples.

In some embodiments, the machine-learning model is trained with training histological images tiles of training histological images from about 1,000 patients. In some embodiments, the patients are different patients. In some embodiments, the machine-learning model is trained with training histological images or training histological image tiles from about 100, 500, 1,000, 2,000, 3,000, 4,000, 5,000, 6,000, 7,000, 8,0000, 9,0000, 10,000, or more patients.

In some embodiments, at least one training histological image from a plurality of training histological images or training histological tiles comes from the same biological sample. In some embodiments, more than one training histological image from a plurality of training histological images or training histological tiles comes from the same biological sample. In some embodiments, at least one training histological image from a plurality of training histological images or training histological tile comes from the same patient. In some embodiments, more than one training histological image from a plurality of training histological images or training histological tile comes from the same patient.

The training histological images may be annotated to generate annotated training histological images. In some embodiments, the training histological images are annotated by a second machine-learning model. In some embodiments, the training histological images are annotated by an individual. In some embodiments, the training histological images or training histological image tiles are annotated by more than one individual. In some embodiments, the individual is human. In some embodiments, the individual is a pathologist (e.g., a trained pathologist), medical professional, biologist, or any other suitable individual capable of identifying mitotic events in a histological image. In some embodiments, crowd sourcing is used to annotate the training histological images or training histological image tiles thereof. In some embodiments, the histological images are annotated by an individual that does not perform the remainder of the machine-learning model training processes. In some embodiments, the histological images are annotated by an individual that is not associated with an entity using the machine-learning model to characterize a disease in a patient.

The individual, such as any of the individuals described above, or the second machine-learning model, may identify normal and abnormal mitotic events in the training histological images. The abnormal mitotic events may represent mitotic events that are characteristics of chromosomal instability as described herein. The abnormal mitotic events may include unaligned metaphase, multipolar pre-anaphase, lagging chromosomes in anaphase, chromosomal bridges in anaphase or telophase, mixed lagging chromosome and chromosomal bridges in anaphase or telophase, and multipolar anaphase or telophase. The normal mitotic events may include prometaphase, normal metaphase, and normal anaphase.

In some embodiments, one or more mitotic events are identified and annotated in the training histological image or training histological image tiles thereof. In some embodiments, any of about 1, 2, 3, 4, 5, 6, 7, 8, 9, 10, or more, normal and/or mitotic events are identified annotated in the training histological image training histological image tiles thereof.

The frequency of abnormal mitotic events in a training histological image or training histological image tiles may be related to the level of chromosome instability present in the histological image. In some embodiments, the training histological images or training histological image tiles differ in the level of chromosome instability (e.g., frequency of abnormal mitotic events) present in the image. The level of chromosomal instability in the training histological images or training histological image tiles may be measured as the number of abnormal mitotic events identified in the training histological image or training histological image tile divided by the total number of mitotic events identified in the training histological image or training histological image tile. In some embodiments, the training histological images or training histological image tiles may contain about 5, 10, 15, 20, 25, 30, 35, 40, 45, 50, 55, 60, 65, 70, 75, 80, 85, 90, 95, or 100, or more, abnormal mitotic events. In some embodiments, the training histological images or training histological image tiles may differ in the level of chromosomal instability by about 0, 5, 10, 15, 20, 25, 30, 35, 40, 45, 50, 55, 60, 65, 70, 75, 80, 85, 90, 95, or 100%. In some embodiments, the level of chromosomal instability metric may be an aggregation of the frequency of each identified mitotic event identified in the input histological image tiles of one input histological image.

In some embodiments, the chromosomal instability may be a numerical chromosomal instability, wherein the abnormal mitotic events result in gains or losses of whole chromosomes. In some aspects, the chromosomal instability may be a structural chromosomal instability, wherein the abnormal mitotic events result in amplification, deletions, inversions, and/or translocations of chromosomal regions, that range in size from single genes to whole chromosomal arms.

For identifying mitotic events, past work has shown, even amongst experts, there may be a considerable amount of label disagreement (Tabata et al., 2019). In some embodiments, labels may be the aggregated result of several labelers (e.g., crowd sourcing and/or expert labelers), imparting a notion of uncertainty. Common aggregations include selecting the majority, computing the average, or taking the union.

In some embodiments, the annotation of the training histological images may take place in two or more phases. The first step may include annotating the location of mitotic events in a training histological image. The second step may include classification of a mitotic event as normal, abnormal, or further classified by the type of abnormality. The separate steps may be completed by the same or different individuals. The individuals may be crowd-sourced or pathologists. In some embodiments, companies can facilitate the gathering of crowd-sourced annotations for medical images.

In some embodiments, the same training histological image is annotated by one or more individuals. In some embodiments, the same training histological images is annotated by 2, 3, 4, 5, 6, or more, individuals. In some embodiments, the one or more individuals do not annotate the image at the same time. In some embodiments, the training histological image is annotated by an expert. In some embodiments, the training histological image is annotated by a non-expert. In some embodiments, the same training histological image is annotated by both an expert (e.g., a trained pathologist) and a non-expert (e.g., an individual that is not an expert in pathology). In some embodiments, the annotations include confidence labels. The term “confidence labels” as used herein refers to labels that describe how certain the individual is that a particular annotation correctly classifies the mitotic event (e.g., anaphase). In some embodiments, the confidence label may classify the mitotic event as “likely”, “certain”, or not occurring. In some embodiments, a “likely” confidence label indicates that the individual annotating the event is greater than about 50% certain of their classification of the mitotic event. In some embodiments, a “certain” confidence label indicates that the individual is about 100% certain of their classification of the mitotic event. In some embodiments, the confidence label may include “certain” normal anaphase, “likely” normal anaphase, “certain” abnormal anaphase, “likely” abnormal anaphase, or non-anaphase. In some embodiments, the annotations from each of the one or more individual annotating the same histological image may be compared. In some embodiments, the confidence labels from the annotations from each of the one or more individual annotating the same histological image may be compared. The concordance of the annotations, optionally including confidence labels, may be assessed to identify a high confidence set of annotations (e.g., annotations that have been given the same classification across different individuals) for use in training the machine-learning model.

A high confidence set of annotations may include object detection annotations that overlap between individuals who have annotated the same histological image. In some embodiments, the high confidence set of annotations comprises a set of annotations wherein a portion of annotations match between different individuals who have annotated the same histological image. For example, the high confidence set of annotations may comprise a set of annotations wherein more than one individual has annotated a mitotic event in the histological image as “abnormal anaphase”. In some embodiments, the high confidence set of annotations comprise a set of annotations wherein at least about 50% of the annotations match between different individuals who have annotated the same histological image, such as at least any of about 60%, 70%, 80%, 90%, 95%, 99%, or more of the annotations match between different individuals who have annotated the same histological image. When confidence labels accompany the annotations, the high confidence set may include histological images wherein zero or more confidence labels use a “likely” classification of a mitotic event, such as any of 1, 2, 3, 4, 5, or more, “likely” classifications of a mitotic event. In some embodiments, the high confidence set includes zero confidence labels that use a “likely” classification of a mitotic event. In some embodiments, the high confidence set may include histological images wherein zero or more confidence labels use a “certain” classification of a mitotic event, such as any of 1, 2, 3, 4, 5, or more, “likely” classifications of a mitotic event. In some embodiments, the high confidence set includes at least one confidence labels that use a “likely” classification of a mitotic event.

In some embodiments, the abnormal mitotic events, such as abnormal anaphase, may be processed with a data augmentation method. The data augmentation method may increase the amount of data by adding slightly modified copies of already existing data or newly created synthetic data from existing data. For example, the data augmentation method may allow for an increased number of abnormal mitotic events (e.g., abnormal anaphase) in a histological image, thereby creating more training data for the machine-learning model. The augmentation method may rotate, distort, and/or zoom in on the histological image. In some embodiments, the augmentation method rotates the histological image. In some embodiments, the augmentation method distorts the histological image. In some embodiments, the augmentation method zooms in on the histological image. The augmentation method may balance the classes of abnormal mitotic events with the total number of mitotic events in a histological image.

In some embodiments, the training histological images include negative instances. Negative instances may be training histological images or training histological images tiles without any mitotic events. In some embodiments, incorporation of negative instances may improve model performance. In some examples, a majority of annotated histological image tiles may not have any mitotic events. In such examples, using the negative instances when calculating a pathological metric (such as a loss function metric) can be important. In some embodiments, the set of training histological images used to train any of the machine-learning models disclosed herein may include at least 1%, least 5%, least 10%, least 15%, least 20%, least 30%, least 40%, least 50%, least 60%, least 70%, least 80%, least 90%, least 95%, least 98%, least 99%, or more, negative instances.

### C. Machine-Learning Model

In some embodiments, the disclosed methods include use one or more machine-learning methods to pre-process and/or analyze histological images (e.g., training histological images, or tiles thereof, and/or input histological images, or tiles thereof). A variety of machine-learning models may be implemented for the disclosed methods. The models may comprise a unsupervised machine-learning model, a weakly-supervised machine-learning model, a human-in-the-loop machine-learning model, a supervised machine-learning model, a deep learning machine-learning model, etc., or any combination thereof.

In some embodiments, unsupervised machine-learning models are used to draw inferences from training histological images and/or training histological image tiles when they are not paired with annotations, in accordance with the methods provided herein. One example of a commonly used unsupervised learning model is cluster analysis. Cluster analysis may be used for exploratory data analysis to find hidden patterns or groupings in multi-dimensional data sets. Other examples of unsupervised learning models include, but are not limited to, artificial neural networks, association rule learning models, etc.

In some aspects, a weakly-supervised machine-learning model is used in accordance with the methods provided herein. In some aspects, these weakly-supervised models may use both annotated and un-annotated training histological images and/or both annotated and un-annotated training histological image tiles.

In some embodiments, a human-in-the-loop model is used in accordance with the methods provided herein. In human-in-the-loop models may include initial iterations of model training that are unsupervised, followed by human interactions to add or modify the computer labels. In some embodiments, humans may add annotations to the model after the model has started to learn from the training histological images and/or training histological image tiles.

In some embodiments, a supervised learning model is used in accordance with the methods provided herein. Supervised learning models may rely on annotated training histological images and/or annotated training histological image tiles. The models may infer the relationships between a set of training histological images and/or training histological image tiles, and user specified annotations for the training histological images and/or training histological image tiles. The training data is used to “teach” the supervised learning model and comprises a set of paired training examples. Examples of supervised learning models include but are not limited to support vector machines (SVMs), artificial neural networks (ANNs), etc.

In some embodiments, Automated Machine-learning (AutoML) can be used to automate model development. In some embodiments, AutoML can be used to automate training and tuning the machine-learning model. In some embodiments, AutoML can be used for a classification model. In some embodiments, AutoML can be used for a regression model.

In some embodiments, artificial neural network algorithms (ANNs) are used in accordance with the methods provided herein. In the context of the present disclosure, ANNs are models inspired by the structure and function of the human brain. ANNs comprise an interconnected group of nodes organized into multiple layers. The ANN architecture may comprise at least an input layer, one or many hidden layers, and an output layer. In some embodiments, the ANN may have about 1, 2, 3, 4, 5, 6, 7, 8, 9, 10, or more hidden layers. Deep learning models are large ANN comprising many layers of coupled “nodes” between the input layer and output layer that may be used, for example, to map histological images or histological image tiles to mitotic events and or chromosomal instability.

The ANN may comprise any total number of layers, and any number of hidden layers, where the hidden layers function as trainable feature extractors that allow mapping of a set of input histological images or input histological image tiles to a preferred output value or set of output values such as identified mitotic events or chromosome instability scores. Each layer of the neural network comprises a number of nodes (or “neurons”). A node receives input that comes either directly from the input data (e.g., histological images, histological image tiles, or mitotic event annotations) or from the output of nodes in previous layers, and performs a specific operation, e.g., a summation operation.

In some cases, a connection from an input to a node is associated with a weight (or weighting factor). In some cases, the node may, for example, sum up the products of all pairs of inputs, Xi, and their associated weights, Wi. In some cases, the weighted sum is offset with a bias, b. In some cases, the output of a neuron may be gated using a threshold or activation function, f, which may be a linear or non-linear function. In some embodiments, the activation function may be, for example, a rectified linear unit (ReLU) activation function or other function such as a saturating hyperbolic tangent, identity, binary step, logistic, arcTan, softsign, parameteric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, can be “taught” or “learned” in a training phase using one or more sets of training histological images. For example, the parameters may be trained using the input data from a training histological image or training histological image tiles a gradient descent or backward propagation method so that the identified mitotic events and/or chromosomal instability metrics computed by the ANN are consistent with the training histological image or training histological image tile examples. The adjustable parameters of the model may be obtained using, e.g., a back propagation neural network training process that may or may not be performed using the same hardware as that used for processing histological images and/or performing biological sample preparation.

Other specific types of deep machine-learning models, e.g., convolutional neural networks (CNNs) (often used for the processing of image data from machine vision systems) may also be used in implementing the disclosed methods. CNNs are commonly composed of layers of different types: convolution, pooling, upscaling, and fully-connected node layers. In some cases, an activation function such as rectified linear unit may be used in some of the layers. In a CNN architecture, there can be one or more layers for each type of operation performed. A CNN architecture may comprise any number of layers in total, and any number of layers for the different types of operations performed. The simplest convolutional neural network architecture starts with an input layer followed by a sequence of convolutional layers and pooling layers, and ends with fully-connected layers. Each convolution layer may comprise a plurality of parameters used for performing the convolution operations. Each convolution layer may also comprise one or more filters, which in turn may comprise one or more weighting factors or other adjustable parameters. In some instances, the parameters may include biases (i.e., parameters that permit the activation function to be shifted). In some cases, the convolutional layers are followed by a layer of ReLU activation function. Other activation functions can also be used, for example the saturating hyperbolic tangent, identity, binary step, logistic, arcTan, softsign, parameteric rectified linear unit, exponential linear unit, softPlus, bent identity, softExponential, Sinusoid, Sine, Gaussian, the sigmoid function and various others. The convolutional, pooling and ReLU layers may function as learnable features extractors, while the fully connected layers may function as a machine-learning classifier. As with other artificial neural networks, the convolutional layers and fully-connected layers of CNN architectures typically include various adjustable computational parameters, e.g., weights, bias values, and threshold values, that are trained in a training phase as described above. In some embodiments, the machine-learning model is any type of CNN model. For example, in some embodiments, the machine-learning model is a RCNN, a Faster-RCNN, or a Mask-RCNN

The machine-learning model architecture may be ResNet, VGG (Simonyan and Zisserman, 2014), or swin-transformer (Liu et al., 2021). These architectures may be used for object detection of mitotic events or classification of normal and/or abnormal mitotic events, such as normal anaphase and/or abnormal anaphase.

For any of the various types of ANN models that may be used in the methods disclosed herein, the number of nodes used in the input layer of the ANN (which enable input of data from, for example, histological images, histological image tiles, a multi-dimensional image feature data set, and/or other types of input data) may range from about 10 to about 20,000 nodes. In some instances, the number of nodes used in the input layer may be at least 10, at least 50, at least 100, at least 200, at least 300, at least 400, at least 500, at least 600, at least 700, at least 800, at least 900, at least 1,000, at least 2,000, at least 3,000, at least 4,000, at least 5,000, at least 6,000, at least 7,000, at least 8,000, at least 9,000, at least 10,000, at least 12,000, at least 14,000, at least 16,000, at least 18,000, or at least 20,000. In some instances, the number of node used in the input layer may be at most 20,000, at most 18,000, at most 16,000, at most 14,000, at most 12,000, at most 10,000, at most 9,000, at most 8,000, at most 7,000, at most 6,000, at most 5,000, at most 4,000, at most 3,000, at most 2,000, at most 1,000, at most 900, at most 800, at most 700, at most 600, at most 500, at most 400, at most 300, at most 200, at most 100, at most 50, or at most 10. Those of skill in the art will recognize that the number of nodes used in the input layer may have any value within this range, for example, about 512 nodes. In some instances, the number of nodes used in the input layer may be a tunable parameter of the ANN model.

In some instances, the total number of layers used in the ANN models used to implement the disclosed methods (including input and output layers) may range from about 3 to about 1,000, or more. In some instances the total number of layers may be at least 3, at least 4, at least 5, at least 10, at least 15, at least 20, at least 40, at least 60, at least 80, at least 100, at least 200, at least 300, at least 400, at least 500, at least 600, at least 700, at least 800, at least 900, or at least 1,000. In some instances, the total number of layers may be at most 1000, at most 800, at most 600, at most 400, at most 200, at most 100, at most 80, at most 60, at most 40, at most 20, at most 15, at most 10, at most 5, at most 4, or at most 3. Those of skill in the art will recognize that, in some cases, the total number of layers used in the ANN model may have any value within this range, for example, 8 layers.

In some instances, the total number of learnable or trainable parameters, e.g., weighting factors, biases, or threshold values, used in the ANN may range from about 10 to about 10,000,000. In some instances, the total number of learnable parameters may be at least 10, at least 100, at least 500, at least 1,000, at least 2,000, at least 3,000, at least 4,000, at least 5,000, at least 6,000, at least 7,000, at least 8,000, at least 9,000, at least 10,000, at least 20,000, at least 40,000, at least 60,000, at least 80,000, at least 100,000, at least 250,000, at least 500,000, at least 750,00, at least 1,000,000, at least 2,500,000, at least 5,000,000, at least 7,500,000, or at least 10,000,000. Alternatively, the total number of learnable parameters may be any number less than 100, any number between 100 and 10,000, or a number greater than 10,000. In some instances, the total number of learnable parameters may be at most 10,000,000, at most 7,500,000, at most 5,000,000, at most 2,500,000, at most 1,000,000, at most 750,000, at most 500,000, at most 250,000, at most 100,000, at most 80,000, at most 60,000, at most 40,000, at most 20,000, at most 10,000, at most 9,000, at most 8,000, at most 7,000, at most 6,000, at most 5,000, at most 4,000, at most 3,000, at most 2,000, at most 1,000, at most 500, at most 100, or at most 10. Those of skill in the art will recognize that the total number of learnable parameters used may have any value within this range, for example, about 2,200 parameters.

In some embodiments, implementation of the disclosed methods may comprise use of an autoencoder model. Autoencoders (also sometimes referred to as an auto-associator or Diabolo networks) are artificial neural networks used for unsupervised, efficient mapping of input data, e.g., training histological images, to an output value, e.g., an image cluster and/or chromosomal instability classifications. Autoencoders are often used for the purpose of dimensionality reduction, i.e., the process of reducing the number of random variables under consideration by deducing a set of principal component variables.

Dimensionality reduction may be performed, for example, for the purpose of feature selection (e.g., selection of the most relevant subset of the image features presented in the original training histological image data set comprising training histological images and/or training histological image tiles) or feature extraction (e.g., transformation of image feature data in the original, multi-dimensional image space to a space of fewer dimensions as defined, e.g., by a series of feature parameters).

Any of a variety of different autoencoder models known to those of skill in the art may be used in the disclosed methods. Examples include, but are not limited to, stacked autoencoders, denoising autoencoders, variational autoencoders, or any combination thereof. Stacked autoencoders are neural networks consisting of multiple layers of sparse autoencoders in which the output of each layer is wired to the input of the successive layer. Variational autoencoders (VAEs) are autoencoder models that use the basic autoencoder architecture, but that make strong assumptions regarding the distribution of latent variables. They use a variational approach for latent representation learning, which results in an additional loss component, and may require the use of a specific training method called Stochastic Gradient Variational Bayes (SGVB).

In some embodiments, implementation of the disclosed methods and systems may comprise the use of a deep convolutional generative adversarial network (DCGAN). DCGANs are a class of convolutional neural networks (CNNs) used for unsupervised learning that further comprise a generative adversarial network (GANs), i.e., they comprise a class of models implemented by a system of two neural networks contesting with each other in a zero-sum game framework. One network generates candidate images (or solutions) and the other network evaluates them. Typically, the generative network learns to map from a latent space (i.e., a representation of compressed data in which similar data points are closer together in space; latent space is useful for learning data features and for finding simpler representations of data for analysis) to a particular data distribution of interest, while the discriminative network discriminates between instances from the true data distribution and the candidate images (or solutions) produced by the generator. The generative network's training objective is to increase the error rate of the discriminative network (i.e., to “fool” the discriminator network) by producing novel synthesized instances that appear to have come from the true data distribution). In practice, a known dataset serves as the initial training data for the discriminator. Training the discriminator involves presenting it with samples from the dataset, until it reaches some level of accuracy. Typically the generator is seeded with a randomized input that is sampled from a predefined latent space (e.g., a multivariate normal distribution). Thereafter, samples synthesized by the generator are evaluated by the discriminator. Backpropagation is applied in both networks so that the generator produces better images, while the discriminator becomes more skilled at flagging synthetic images. The generator is typically a deconvolutional neural network, and the discriminator is a convolutional neural network. In some instances, implementation of the disclosed methods and systems may comprise the use of a Wasserstein generative adversarial network (WGAN), a variation of the DCGAN structure that uses a slightly modified architecture and/or a modified loss function.

In some embodiments, the disclosed methods may use a cluster method to cluster histological images or histological image tiles according to features. Any variety of clustering methods known to those skilled in the art may be used. Clustering methods may include, but are not limited to, k-means clustering methods, hierarchical clustering methods, mean-shift clustering methods, density-based spatial clustering methods, expectation-maximization clustering methods, and mixture model (e.g., mixtures of Gaussians) clustering methods.

In some embodiments, hierarchical clustering method may be used to group histological images or histological image tiles into groups or clusters. In some embodiments, a feature space (e.g., a mitotic event) in a histological image or histological image tiles is clustered. Hierarchical clustering methods may be used to identify a set of clusters such as histological images with similar normal or abnormal mitotic events. Initially, each data point is treated as a separate cluster. A distance matrix for pairs of data points is calculated, and the method then repeats the steps of: (i) identifying the two clusters that are closest together, and (ii) merging the two most similar clusters. The iterative process continues until all similar clusters have been merged.

In some embodiments, a clustering model may use a Gaussian mixture model. Gaussian mixture models are probabilistic models that assume all data points (e.g., histological image or histological image segment) in a data set may be represented by a mixture of a finite number of Gaussian distributions with unknown peak height, position, or standard deviations. The approach is similar to generalizing a k-means clustering method to incorporate information about the covariance structure of the data as well as the centers of the latent Gaussians.

In some embodiments, any of the machine-learning models provided herein models may be used for object detection to identify mitotic events. Object detection models may be trained with the coordinates of annotated mitotic events in the training histological images. The coordinates may correspond to specific regions on a training histological image or image tile, e.g. [x₁, y₁, w, h]. The object detection model may be Faster-RCNN (Ren et al. (2015)) which uses a CNN to predict location and class of boxes. Additionally, the model may be trained with smoothed L1 loss for positions of the event and cross-entropy loss for the objectiveness and the class of bounding events. In some embodiments, a RCNN model is used for mitosis detection. In some embodiments, the Faster-RCNN model or other related RCNN methods are used for mitosis detection, such as Mask-RNN. RCNN methods consider objects, such as mitotic events, as anchors during object detection. In some embodiments, anchor-based detection methods require two-stage object detection.

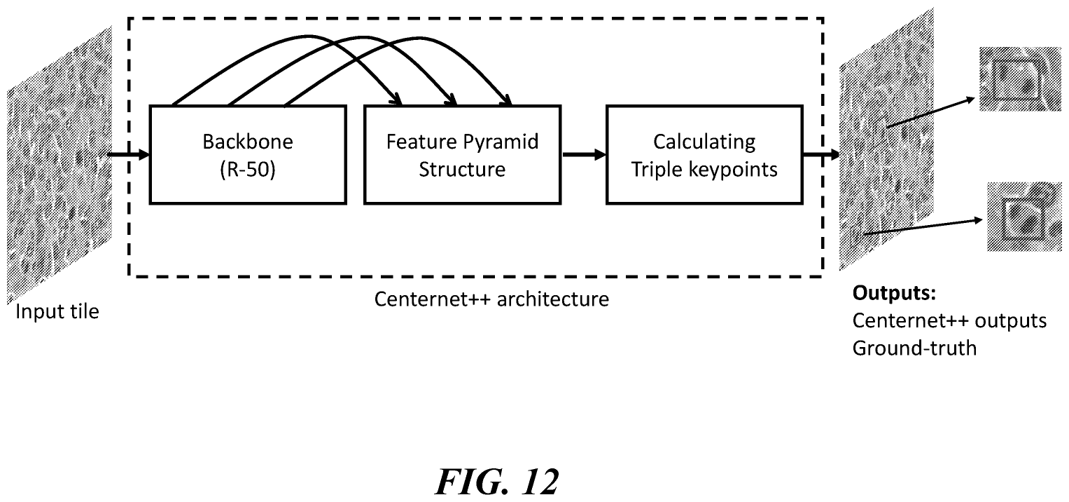

In some embodiments, a CenterNet++ model is used for mitosis detection (e.g., object detection). In some embodiments, a CenterNet++ model is used for anaphase figure detection. An exemplary CenterNet++ architecture for detection of anaphase figures in stained input histological images is shown in FIG. 12.

CenterNet++ may be useful for the detection of anaphase figures because it considers objects (e.g., anaphase figures) as triplet keypoints. In some embodiments, classifying mitotic events as triplet keypoints improves precision and recall of the machine-learning model. In some embodiments, the CenterNet++ model does not require the model to find a predefined bounding box to detect a mitotic event. In some embodiments, the CenterNet++ model does not require two-stage object detection. In some embodiments, the CenterNet++ model has a decreased number of false positive bounding boxes compared to alternative object detection methods.

Machine-learning models may be used for classification of detected mitotic events. The classification model may take an annotated training histological image or training histological image tile as input and output a continuous value that be converted to binary based on a threshold to deterring presence of a mitotic event. Regression models may be used to predict confidence of a mitotic event in an input histological image or image tile. The model may be generated by replacing a last layer of a binary classification architecture with a layer generating a scaler output. The model may be trained using mean-squared error loss. In some aspects, object detection and classification models can be evaluated on some set of metrics derived from the confusion matrix, and, in the case of object detection, conditioned on the intersection over union of the predicted and ground truth labels. In some embodiments, because the number of instances is not bounded in the object detection setting, the mean (over the population) average (in the image) metric can be reported.

Any of a variety of commercial or open-source program packages, program languages, or platforms known to those of skill in the art may be used to implement the machine-learning models of the disclosed methods and systems. Examples include, but are not limited to, Shogun (www.shogun-toolbox.org), Mlpack (www.mlpack.rog), R (r-project.org), Weka (www.cs.waikato.ac.nz/ml/weka/), Python (www.python.org), and/or Matlab (MathWorks, Natick, MA).

In some embodiments, cross-entropy loss is used in training the machine-learning model. In some embodiments, cross-entropy loss is used as a loss function when optimizing the machine-learning model. In some embodiments, cross-entropy loss is particularly used in logistic regression and artificial neural network machine-learning models.

### D. Statistical Data Analysis

The disclosed methods may comprise use of statistical data analysis techniques. In some embodiments, the statistical data analysis techniques are used to assign confidence to a mitotic event identified in an input histological image and/or input histological images tiles. In some embodiments, statistical data analysis techniques include processes for input histological image and/or input histological images tiles processing and/or methods incorporated into the machine-learning model to identify key components that underlie observed mitotic event variation. The combination of one or more statistical analysis models, e.g., principal component analysis (PCA), may be used in combination with a machine-learning model, to identify key components (or attributes) that comprise mitotic event heterogeneity in histological images. In some embodiments, the basis set of key attributes identified by a statistical and/or machine-learning-based analysis may comprise about 1 key attribute, 2 key attributes, 3 key attributes, 4 key attributes, 5 key attributes, 6 key attributes, 7 key attributes, 8 key attributes, 9 key attributes, 10 key attributes, 15 key attributes, 20 key attributes, or more.

Any of a variety of suitable statistical analysis methods known to those of skill in the art may be used in performing the disclosed methods. Examples include, but are not limited to, principal component and other eigenvector-based analysis methods, regression analysis, probabilistic graphical models, or any combination thereof. In some embodiments, the statistical analysis method is a probability metric for a predicted classification of a mitotic event (e.g., annotated mitotic event that may be annotated abnormal mitotic events or annotated normal mitotic events). In some embodiments, the probability metric for a predicted classification of the annotated mitotic event is with confidence above a specified threshold value. In some embodiments, the annotated mitotic events with confidence above a specified threshold are used to compute a chromosomal instability metric corresponding to the biological sample in the input histological image or input histological image tiles. In some embodiments, the chromosomal instability metric is the frequency of abnormal mitotic events with confidence above a specified threshold compared to all mitotic events in an input histological image or input histological image tile.

### F. Machine-Learning Model Output

In some embodiments, the machine-learning model outputs a chromosomal instability metric (e.g., chromosomal instability score). In some embodiments, the chromosomal instability metric is one or more chromosomal instability metrics. In some embodiments, the output comprises an output of levels of different types or subtypes of chromosomal instability. In some embodiments, the output comprises an output of multiple chromosomal instability metrics to measure different types of chromosomal instability, such as any of the types of chromosomal instability described herein. In some embodiments, the chromosomal instability metric may be a frequency of each identified mitotic event (e.g., abnormal mitotic event) in an input histological image. In some embodiments, the chromosomal instability metric may be a frequency of each identified mitotic event (e.g., abnormal mitotic event) in the input histological image tile. In some embodiments, the chromosomal instability metric may be an aggregation of the frequency of each identified mitotic event (e.g., abnormal mitotic event) identified in the tiles of a whole input histological image. In some embodiments, the chromosomal instability metric may be the probability that the mitotic event is abnormal. In some embodiments, the chromosomal instability metric may be the number of abnormal mitotic events normalized by the tumor area.

In some embodiments, the machine-learning model output does not require human intervention. Thus, the machine-learning model described herein may be fully automated. In some embodiments, the machine-learning model uses transfer learning and feature aggregation to accurately discriminate a pathological status, such as chromosome instability metric, of a biological sample of histopathology slides (e.g., input histological images or input histological image tiles) without human intervention. In some embodiments, the predicted pathological status is a predicted chromosome instability metric of the biological sample in the input histological image or input histological image tiles, or any combination thereof. In some embodiments, the predicted chromosomal instability metric is the frequency of abnormal mitotic events with confidence above a specified threshold compared to all mitotic events in an input histological image. In some embodiments, the predicted chromosomal instability metric is the frequency of abnormal mitotic events with confidence above a specified threshold compared to all mitotic events in an input histological image tile.

A predicted chromosomal instability metric of in input histological image or input histological image tiles may be displayed to a user. In some embodiments, the predicted chromosomal instability metric is displayed as an output of the machine-learning model described herein. In some embodiments, the predicted chromosomal instability metric is displayed on the input histological images or input histological image tiles. In some embodiments, the predicted chromosomal instability metric is a patch-level prediction, wherein the predicted pathological status metric is computed on each input histological image tile. Patch-level predicted chromosomal instability metrics may reveal intra-image heterogeneity (e.g., intra-tumor heterogeneity of chromosomal instability). Thus, in some embodiments, the biological sample of the input histological image or input histological image tiles comprise different patch-level predicted chromosomal instability metrics. The patch-level chromosomal instability metrics may be averaged to determine the predicted chromosomal instability metric of the biological sample of a whole inputted histological image. In some embodiments, the biological sample of the whole input histological image is predicted to be “high chromosomal instability” status or “low chromosomal instability” status using the predicted chromosomal instability metric. In some embodiments, the chromosomal instability status of the biological sample of the input histological images or input histological image tiles is used to characterize a disease (e.g., cancer). For example, a biological sample predicted to have high chromosomal instability status may be more likely to have advanced disease (e.g., cancer) pathology compared to a biological sample predicted to have low chromosomal instability.

In some embodiments, the machine-learning model outputs a chromosomal instability metric. In some embodiments, the chromosomal instability metric may be a proportion of abnormal mitotic events identified in the input histological image. In some embodiments, the chromosomal instability metric may be the proportion of abnormal mitotic events identified in the input histological image tiles. In some embodiments, the chromosomal instability metric may be a proportion calculated as an aggregation of the abnormal mitotic events identified in the tiles of a whole input histological image.

In some embodiments, the chromosomal instability metric may be a proportion of unaligned metaphase events identified in the input histological image. In some embodiments, the chromosomal instability metric may be the proportion unaligned metaphase events identified in the input histological image tiles. In some embodiments, the chromosomal instability metric may be a proportion calculated as an aggregation of the unaligned metaphase events identified in the tiles of a whole input histological image.

In some embodiments, the chromosomal instability metric may be a proportion of multipolar pre-anaphase events identified in the input histological image. In some embodiments, the chromosomal instability metric may be the proportion of multipolar pre-anaphase events identified in the input histological image tiles. In some embodiments, the chromosomal instability metric may be a proportion calculated as an aggregation of the multipolar pre-anaphase events identified in the tiles of a whole input histological image.

In some embodiments, the chromosomal instability metric may be a proportion of anaphase lagging chromosomes events identified in the input histological image. In some embodiments, the chromosomal instability metric may be the proportion of anaphase lagging chromosomes events identified in the input histological image tiles. In some embodiments, the chromosomal instability metric may be a proportion calculated as an aggregation of the anaphase lagging chromosomes events identified in the tiles of a whole input histological image.

In some embodiments, the chromosomal instability metric may be a proportion of anaphase chromosomal bridges events identified in the input histological image. In some embodiments, the chromosomal instability metric may be the proportion of anaphase chromosomal bridges events identified in the input histological image tiles. In some embodiments, the chromosomal instability metric may be a proportion calculated as an aggregation of the anaphase chromosomal bridges events identified in the in the tiles of a whole input histological image.

In some embodiments, the chromosomal instability metric may be a proportion of mixed anaphase lagging chromosome and chromosomal bridges events identified in the input histological image. In some embodiments, the chromosomal instability metric may be the proportion of mixed anaphase lagging chromosome and chromosomal bridges events identified in the input histological image tiles. In some embodiments, the chromosomal instability metric may be a proportion calculated as an aggregation of the mixed anaphase lagging chromosome and chromosomal bridges identified in the tiles of a whole input histological image.

In some embodiments, the chromosomal instability metric may be a proportion of multipolar anaphase events identified in the input histological image. In some embodiments, the chromosomal instability metric may be the proportion of multipolar anaphase events identified in the input histological image tiles. In some embodiments, the chromosomal instability metric may be a proportion calculated as an aggregation of the multipolar anaphase identified in the tiles of a whole input histological image.

In some embodiments, the chromosomal instability metric may be a proportion of normal mitotic events identified in the input histological image. In some embodiments, the chromosomal instability metric may be the proportion of normal mitotic events identified in the input histological image tiles. In some embodiments, the chromosomal instability metric may be a proportion calculated as an aggregation of the normal mitotic events identified in the tiles of a whole input histological image.

In some embodiments, the chromosomal instability metric may be the number of abnormal mitotic events normalized by the tumor size. In some embodiments, the tumor size can come from the tumor segmented image. The tumor size may be tumor area calculated after removing non-tumor area, such as stroma and fat, from whole slide images or image tiles.

In some embodiments, the chromosomal instability metric may be calculated with only the high quality mitotic events identified in the inputted histological image or inputted histological image segment. In some embodiments, the chromosomal instability metric is displayed to a user.

### G. Processors, Computer Systems, and Distributed Computer Environments

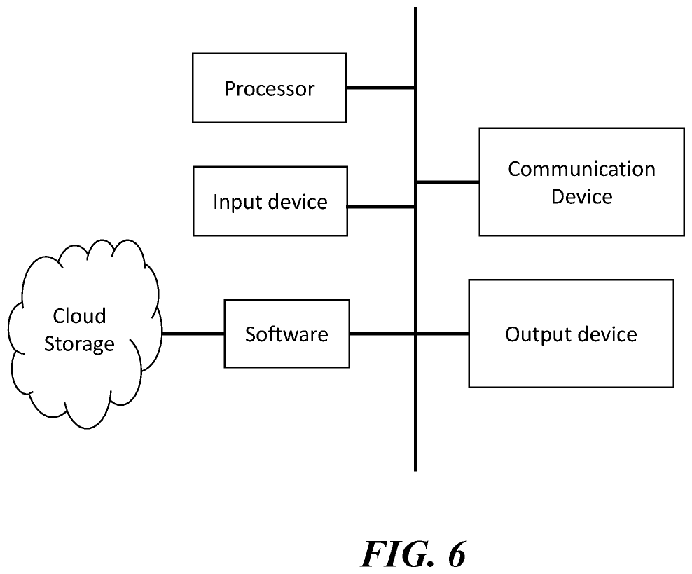

In some embodiments, one or more processors may be used to implement the machine-learning-based methods and systems disclosed herein. In addition to running the machine-learning and/or statistical analysis methods used to implement the disclosed methods, the one or more processors may be used for inputting data (e.g., input histological images and/or input histological image tiles), to the machine-learning model, or for outputting a result from the machine-learning model. The one or more processors may comprise a hardware processor such as a central processing unit (CPU), a graphic processing unit (GPU), a general-purpose processing unit, or other computing platform such as a cloud based computing platform. An exemplary cloud based computing platform according to some embodiments is illustrated in FIG. 6. The processor may be comprised of any of a variety of suitable integrated circuits, microprocessors, logic devices, field programmable gate arrays (FPGAs), and the like. Although the disclosure is described with reference to a processor, other types of integrated circuits and logic devices are also applicable. The processor may have any suitable data operation capability. For example, the processor may perform about 512 bit, 256 bit, 128 bit, 64 bit, 32 bit, or 16 bit data operations.

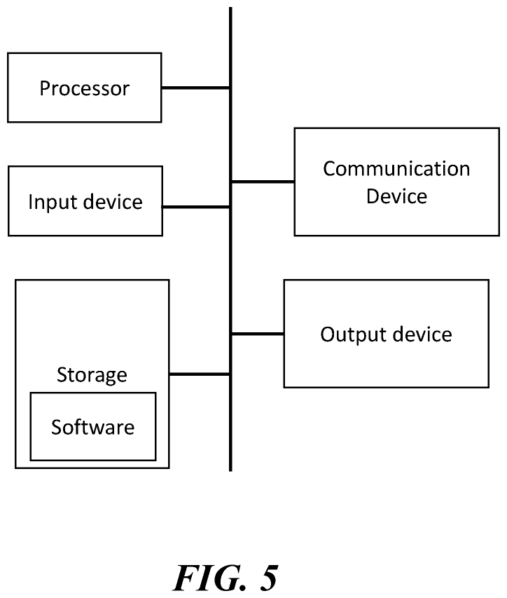

FIG. 5 illustrates an example computer system in accordance with one or more examples described herein. As shown in FIG. 5, a computer system may comprise one or more processors, an input device, an output devices, storage, and a communication device. The computer system may be any suitable type of microprocessor-based device, such as a personal computer, workstation, server, or handheld device. The computer components described herein may be connectable or integrated into the computer.

In some embodiments, the input device may be any suitable device that provides input, such as a touch screen, keyboard or keypad, mouse, or voice-recognition device. In some aspects, the output device can be any suitable device that provides output, such as a touch screen, haptics device, or speaker. In some embodiments, the storage can be any suitable device that provides storage, such as an electrical, magnetic, or optical memory including RAM, cache, hard drive, or removable storage disk. In some embodiments, storage can be in the form of an external computing cloud. The communication device may be any suitable device capable of transmitting and receiving signals over a network, such as a network interface chip or device.

In some embodiments, a computer-readable storage medium may store programs with the instructions for one or more processor of an electronic device to run the machine-learning model disclosed herein. The computer-readable storage medium disclosed herein can be stored in memory or storage and can be executed by a processor. The computer-readable storage medium can be stored or propagated for use by or in connection with an instruction execution system, apparatus, or device, such as those described above, that can fetch instructions associated with the program from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

The computer system described herein may be connected to a network, which can be any suitable type of interconnected communication system. The network can implement any suitable communication protocol and can be secured by any suitable security protocol. The network may comprise network links of any suitable arrangement that can implement the transmission and reception of network signals, such as wireless network connections, T1 or T3 lines, cable networks, DSL, or telephone lines.

The computer system described herein may implement any operating system for operating on the network. The computer-readable storage medium can be written in any suitable programming language, such as C, C++, Java, or Python. In some embodiments, programs embodying the functionality of the present disclosure can be deployed in different configurations, such as in a client/server arrangement or through a web browser as a web-based application or web service, for example.

In some embodiments, the methods and systems of the present disclosure may be implemented through 1, 2, 3, 4, 5, or more, machine-learning models. A machine-learning model can be implemented by way of a coded program instructions upon execution by the central processing unit.

### IV. System

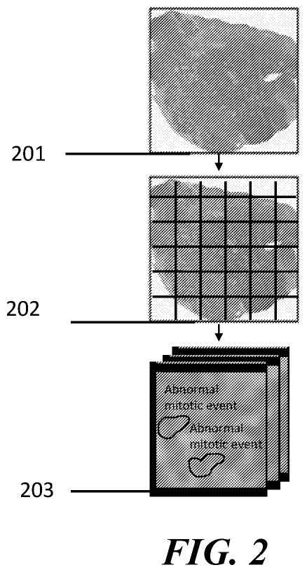

One aspect of the present application provides a system for using the methods described herein to train a machine-learning model using annotated training histological images and/or annotated training histological image tiles. FIG. 2 illustrates a system for preparing an annotated training histological image. A hematoxylin and eosin (H&E) stained histological image of a tumor is obtained, 201. The training histological image is segmented into individual tiles of the training histological image, 202. Mitotic events (e.g., abnormal and normal mitotic events) are identified in the training histological image tiles, and the type of mitotic event along with the identification are annotated in the training histological image tiles to generate annotated training histological image tiles, 203. These annotated training histological image tiles can be used to train the machine-learning models described herein.

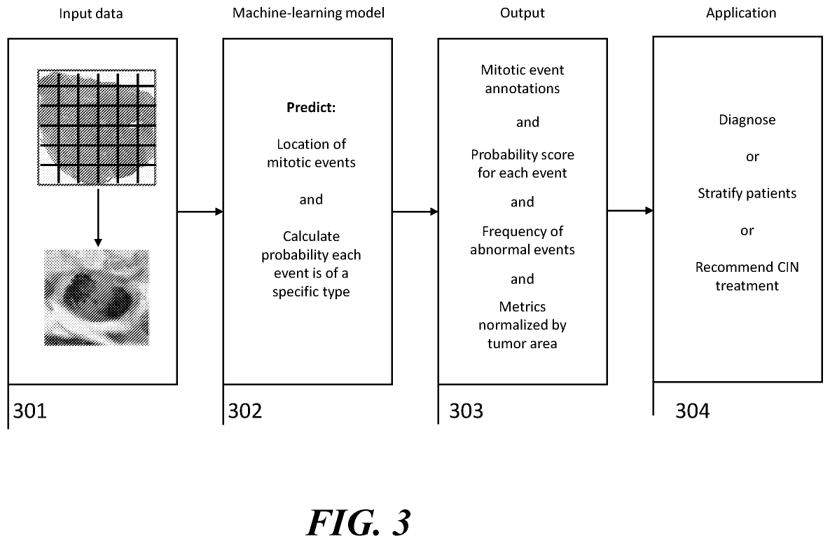

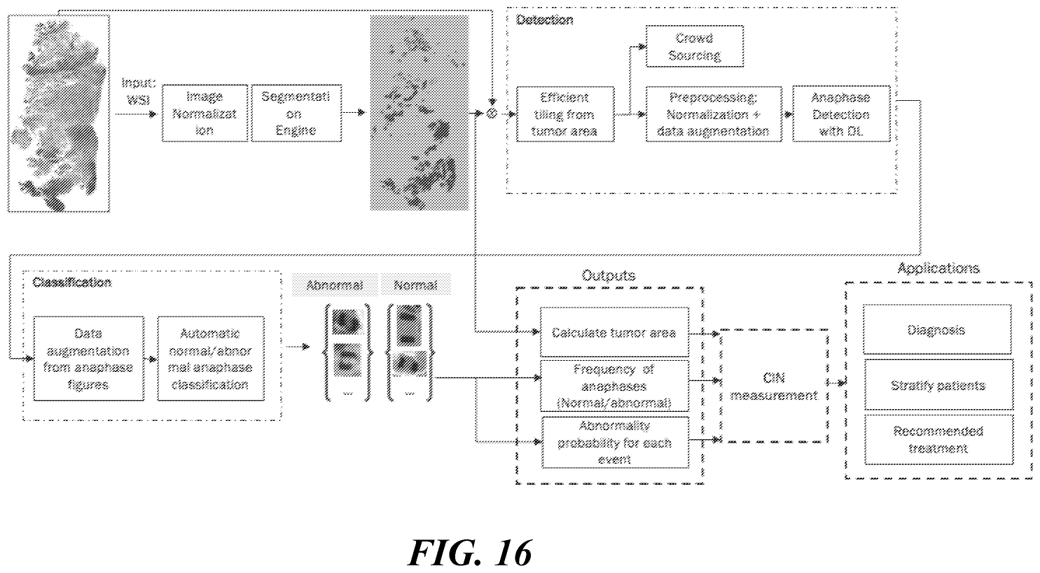

One aspect of the present application provides systems for using the methods described herein to characterize a disease in a patient using input histological images and/or input histological image tiles and a trained machine-learning model. FIG. 3 illustrates a system for characterizing a disease in a patient using the machine-learning methods described herein. An input histological image, or input histological image tiles thereof, or a biological sample are obtained, and are inputted into the trained machine-learning model, 301. In some embodiments, the input histological image or input histological image tiles thereof are stained with H&E. In some embodiments, the method further comprises staining the biological sample with H&E to generate a stained biological sample. In some embodiments, the method further comprises imaging the stained (e.g., H&E stained) biological sample using any of the imaging techniques described herein. In some embodiments, the biological sample comprises a tumor sample. In some embodiments, the biological sample comprises a tumor sample obtained from a patient (e.g., a patient tumor slice obtained from a biopsy). The machine-learning model predicts the location of individual mitotic events within the histological image or image tiles, 302. In some embodiments, image segmentation may be used to delete non-tumor areas, such as stroma and fat, in order to calculate tumor area. The mitotic events may be normal or abnormal as described above. The machine-learning model also assigns a probability to each mitotic event identified, for use in the model output, 302. The machine-learning model is trained to output chromosomal instability metrics, such as the types of mitotic events (e.g., types of abnormal mitotic events and normal mitotic events) identified in the input histological image, or input histological image tiles thereof, the associated probability for each annotation, and the frequency of abnormal events calculated as the number of abnormal mitotic events normalized by the number of normal mitotic events in the input histological image or input histological image tiles thereof, and/or one of the above metrics normalized by the tumor area, 303. The output may be displayed to a user who can use the chromosomal instability metrics for a variety of tasks, such as, to diagnose a patient, stratify multiple patients based on the level of chromosomal instability identified in their biological samples, characterize disease status, and/or to recommend specific treatments, diagnose, or understand prognosis, as described herein, 304. See, also, FIG. 16 which described the method in greater detail.

One aspect of the present application provides systems for implementing the methods described herein. In some embodiments, the disclosed systems may comprise one or more processors or computer systems, one or more memory devices, and one or many programs, where the one or more programs are stored in the memory devises and contain instructions which, when executes by one or more processors, cause the system to perform the method for training a machine-learning model to detect chromosomal instability from input histological images, or input histological image tiles thereof, or the method for characterizing a disease using a machine-learning model able to detect chromosomal instability from input histological images, or from input histological image tiles thereof, as described herein. In some aspects, the disclosed system may further comprise one or more display devices (e.g., monitors), one or more imaging units (e.g., bright-field microscope, florescent microscope), one or more output devices (e.g., printers), one or more computer network interface devices, or any combination thereof.

In some embodiments, the performance of the disclosed methods and systems is assessed. In some embodiments, assessing the methods and systems includes determining the area under a receiver operating characteristic curve (AUROC) on either a per histological image basis or after the aggregation of results from multiple histological images or histological image tiles. A receiver operating characteristic (ROC) curve is a graphical plot of the classification model's performance as its discrimination threshold is varied. In some instances, the performance of the disclosed methods and systems is characterized by an AUROC value of at least 0.50, at least 0.55, at least 0.60, at least 0.65, at least 0.70, at least 0.75, at least 0.80, at least 0.85, at least 0.90, at least 0.91, at least 0.92, at least 0.93, at least 0.94, at least 0.95, at least 0.96, at least 0.97, at least 0.98, or at least 0.99. In some instances, the performance of the disclosed methods and systems is characterized by an AUROC of any value within the range of values described in this paragraph, e.g., an AUROC value of 0.876. In some instances, the performance of the disclosed methods and systems vary depending on the specific training histological images for which the classification models are trained.

In some embodiments, the performance of the disclosed methods and systems is assessed by evaluating the clinical sensitivity and specificity for correctly identifying mitotic events in an inputted histological image or identifying a level of chromosomal instability in an inputted histological image. In some instances, the clinical sensitivity (e.g., how often the method correctly identifies mitotic events or the level of chromosomal instability, as calculated from the number of true positive results divided by the sum of true positive and false negative results), may be at least 50%, at least 60%, at least 70%, at least 75%, at least 80%, at least 85%, at least 90%, at least 95%, at least 98%, at least 99%, or at least 99.9%. In some instances, the clinical specificity (e.g., how often the method correctly identifies mitotic events or the level of chromosomal instability, as calculated from the number of true negatives divided by the sum of false positives and true negatives) may be at least 50%, at least 60%, at least 70%, at least 75%, at least 80%, at least 85%, at least 90%, at least 95%, at least 98%, at least 99%, or at least 99.9%. In some instances, adjustment of a threshold is used to distinguish between positive and negative results in tradeoffs between clinical sensitivity and clinical specificity. For example, the threshold may be adjusted to increase clinical sensitivity with a concomitant decrease in clinical specificity, or vice versa.

In some embodiments, the performance of the disclosed methods and systems may be assessed by the positive predictive value (PPV) of the disclosed methods and systems (e.g., the percentage of positive results that are true positives as indicated by a pathologist annotation or other reference method) is calculated as the number of true positives divided by the sum of true positives and false positives. The PPV may be at least 70%, at least 75%, at least 80%, at least 85%, at least 90%, at least 95%, at least 98%, at least 99%, or at least 99.9%. In some embodiments, the negative predictive value (NPV) of the disclosed methods and systems (e.g., the percentage of negative results that are true negatives as indicated by a pathologist annotation or other reference method) is calculated from the number of true negatives divided by the sum of false negatives and true negatives can be used to assess the model performance. The NPV may be at least 70%, at least 75%, at least 80%, at least 85%, at least 90%, at least 95%, at least 98%, at least 99%, or at least 99.9%.

### V. Applications of the Methods

In some aspects, the provided embodiments can be applied in a method for characterizing a disease in an individual, such as characterizing a cancer, for example from intact tissues or samples (e.g., a tumor resection) in which a chromosomal instability metric has been determined using the provided machine-learning models. In some aspects, the embodiments can be applied to a diagnostic or prognostic method. In some aspects, the provided embodiments can be used to inform treatment selection (e.g., selection of a pharmaceutical drug) and/or evaluate the efficacy of a treatment, to further characterize a disease (e.g., cancer) in the individual.

In some embodiments, the individual is a mammal (e.g., human, non-human primate, rat, mouse, cow, horse, pig, sheep, goat, dog, cat, etc.). In some embodiments, the individual is a human. The individual may be a clinical patient, a clinical trial volunteer, or an experimental animal. In some embodiments, the individual is younger than about 60 years old (including, for example, younger than about any of 50, 40, 30, 25, 20, 15, 10 years old, or younger). In some embodiments, the individual is older than about 60 years old (including, for example, older than about any of 70, 80, 90, 100 years old), or older. In some embodiments, the individual is diagnosed with, or genetically prone to, one or more of the diseases or disorders described herein (such as a cancer). In some embodiments, the individual has one or more risk factors associated with one or more diseases or disorders described herein. For example, the individual may be a patient suspected of having a disease, such as cancer. In some embodiments, the patient has been previously diagnosed with a disease prior to the application of a provided machine-learning model. In some embodiments, the patient has been previously diagnosed with cancer prior to the application of a provided machine-learning model.

A disease is characterized based on the determined chromosomal instability metric obtained using a machine-learning model described herein. In some embodiments, input histological images of a biological sample (e.g., a tumor sample from a patient) are inputted into a trained machine-learning model trained using a plurality of pathologist annotated training histological images. The pathological status of the one or more input histological images may be classified based on a determined frequency of abnormal mitotic events, such as any of the abnormal mitotic events described herein. In some embodiments, the annotated mitotic events are used to compute a chromosomal instability metric for the biological sample of the input histological images. The chromosomal instability metric is then used to characterize a disease in the patient.

The disease is characterized using information (e.g., a chromosomal instability metric) obtained from a provided machine-learning model may include, but is not limited to, cancer, central nervous system (CNS) disease, autoimmune disease, Fanconi anaemia, Nijmegen breakage syndrome, Bloom syndrome, ataxia telangiectasia, ataxia telangiectasia-like disorder, immunodeficiency/centromeric instability/facial anomalies syndrome, Cockayne syndrome, trichothiodystrophy, xeroderma pigmentosum, DNA ligase I deficiency, PMS2 deficiency, and DNA recombinase repair defects (e.g., DNA-PKcs, Artemis, DNA ligase 4, Cernunnos/XLF). In some embodiments, the cancer includes, but is not limited to, acute lymphoblastic leukemia (ALL), acute myeloid leukemia (AML), bladder cancer, breast cancer, brain cancer, cervical cancer, colon cancer, colorectal cancer, endometrial cancer, gastrointestinal cancer, glioma, glioblastoma, head and neck cancer, kidney cancer, liver cancer, lung cancer, lymphoid neoplasia, melanoma, a myeloid neoplasia, ovarian cancer, pancreatic cancer, prostate cancer, squamous cell carcinoma, testicular cancer, stomach cancer, or thyroid cancer. In some instances, a cancer includes a lymphoid neoplasia, head and neck cancer, pancreatic cancer, endometrial cancer, colon or colorectal cancer, prostate cancer, glioma or other brain/spinal cancers, ovarian cancer, lung cancer, bladder cancer, melanoma, breast cancer, a myeloid neoplasia, testicular cancer, stomach cancer, cervical, kidney, liver, or thyroid cancer.

In some embodiments, the provided methods are applicable to a diagnostic method for diagnosis of a disease in a patient. For example, a provided machine-learning model may be used to diagnose cancer in the patient, wherein the chromosomal instability metric may be used to diagnose and/or predict the occurrence of a disease in the patient. In some embodiments, the chromosomal instability metric of the input histological images are indicative of a disease profile in the patient when the frequency of abnormal mitotic events is increased compared to all identified mitotic events in an inputted histological images. For example, a cancer disease profile may comprise an increased frequency of unaligned metaphase, multipolar pre-anaphase, lagging chromosomes in anaphase, chromosomal bridges in anaphase or telophase, mixed lagging chromosome and chromosomal bridges in anaphase or telophase, and multipolar anaphase or telophase, abnormal mitotic events in an input histological image. In some embodiments, the patient is diagnosed with a cancer based on the chromosomal instability metric of the input histological images determined using a provided machine-learning model.

In some embodiments, the chromosomal instability metric, determined using a provided machine-learning model, is used for predicting disease prognosis in a patient. Disease prognosis is determined in patients that have already developed the disease of interest, such as cancer. The term “disease prognosis” as used herein refers to a prediction of the possible outcomes of a disease (e.g., death, chance of recovery, and/or recurrence) and the frequency with which these outcomes can be expected to occur. The prognosis of a disease may be predicted based on the degree of disease progression in the patient. In some embodiments, the disease is cancer, and a cancer prognosis is predicted in a patient using a chromosomal instability metric determined using a machine-learning model described herein. Additional factors contributing to disease prognosis, such as demographics (e.g., age), behavior (e.g., alcohol consumption, smoking), and co-morbidity (e.g., other conditions accompanying the disease in question), may be evaluated in combination with the chromosomal instability metric. Cancer-specific factors contributing to disease prognosis in combination with the chromosomal instability metric may include, but are not limited to, tumor size, high white cell count, cancer type, and the location of the cancer within the patient's body.

In some cases, the disease (e.g., cancer) is further classified by a grade for prognostic purposes. For example, the grades of cancer include Grade X, Grade 1, Grade 2, Grade 3 or Grade 4. In some embodiments, the cancer grade is determined based on the chromosomal instability metric of the input histological images. In some embodiments, the cancer grade is further indicated by a category of tubule formation, nuclear grade and/or the mitotic rate. In some cases, a cancer stage is assigned based on the tumor, the regional lymph nodes and/or distant metastasis. For example, the stages assigned to the tumor include TX, T0, Tis, T1, T2, T3, or T4. For example, the stages assigned to a regional lymph node include NX, N0, N1, N2, or N3. For example, the stages assigned to a distant metastasis include MX, M0, or M1. In some embodiments, the stages include stage 0, stage I, stage II, stage III or stage IV. Sometimes, the cancer is classified as more than one grade or stage of cancer. In some embodiments, a chromosomal instability metric can be correlated to different stages of tumor development, such as initiation, promotion, and progression. In some embodiments, the grade and stage of the cancer is used to predict disease prognosis in combination with the chromosomal instability metric determined using a provided machine-learning model.

In some embodiments, the method further comprises implementing a treatment regimen based on the disease diagnosis and/or prognosis of the disease. In some embodiments, the treatment regimen comprises treatment with a drug that specifically targets chromosomal instability in a patient. In some embodiments, the drug is a pharmaceutical composition. For example, the drug may target abnormal mitotic structures. In some embodiments, drugs with various mechanisms of action, such as anti-microtubule activity, histone deacetylase inhibition, mitotic checkpoint inhibition, and targeting of DNA replication and damage responses, may serve as effective treatment for chromosomal instability. Drugs effective for the treatment of a cancer described herein include proteasome inhibitors, immunomodulatory drugs, glucocorticoids, and conventional chemotherapeutics. In some embodiments, drug treatment is combined with autologous stem cell transplantation if the patient is eligible.

The amount of the drug (e.g., pharmaceutical composition) administered to a patient may vary with the particular composition, the method of administration, and the particular type of disease being treated. The amount should be sufficient to produce a desirable beneficial effect. For example, in some embodiments, the amount of the drug is effective to result in an objective response (such as a partial response or a complete response). In some embodiments, the amount of drug is sufficient to result in a complete response in the individual. In some embodiments, the amount of the drug is sufficient to result in a partial response in the individual. In some embodiments, the amount of the drug administered alone is sufficient to produce an overall response rate of more than about any one of 40%, 50%, 60%, or 64% among a population of patients treated with the composition. Responses of the patients to the treatment can be determined, for example, based on the chromosomal instability metric of the methods described herein.

In another embodiment, described herein are methods for determining the therapeutic efficacy of a pharmaceutical drug. These methods are useful in informing choice of the drug for a patient, as well as monitoring the progress of a patient on the drug.

Treatment of a patient involves administering the drug, or a combination of drugs, in a particular regimen. In some instances, the regimen involves a single dose of the drug, a combination of drugs, and/or multiple doses of the drug over time. The doctor or clinical researcher monitors the effect of the drug on the patient or subject over the course of administration. If the drug has a pharmacological impact on the condition, the profile of a chromosomal instability metric of the biological sample of the input histological images of the present invention are changed toward a non-cancer profile (e.g., decreased frequency of abnormal mitotic events). In some embodiments, the non-cancer profile comprises a decreased frequency of unaligned metaphase, multipolar pre-anaphase, lagging chromosomes in anaphase or telophase, chromosomal bridges in anaphase or telophase, mixed lagging chromosome and chromosomal bridges in anaphase or telophase, and multipolar anaphase, abnormal mitotic events in an input histological image.

In some embodiments, the drug is administered in an amount sufficient to decrease the size of a tumor, decrease the number of cancer cells, or decrease the growth rate of a tumor by at least about any one of 10%, 20%, 30%, 40%, 50%, 60%, 70%, 80%, 90%, 95% or 100% compared to the corresponding tumor size, number of cancer cells, or tumor growth rate in the same subject prior to treatment or compared to the corresponding activity in other subjects not receiving the treatment. Standard methods can be used to measure the magnitude of this effect, such as in vitro assays with purified enzyme, cell-based assays, animal models, or human testing. In some embodiments, the drug is administered in an amount sufficient to decrease the frequency of abnormal mitotic events in a histological image by at least about any one of 10%, 20%, 30%, 40%, 50%, 60%, 70%, 80%, 90%, 95% or 100% compared to the corresponding abnormal mitotic events in the same subject prior to treatment or compared to the corresponding activity in other subjects not receiving the treatment.

In some instances, the chromosomal instability metric in the patient is followed during the course of treatment. In some embodiments, the patient is re-biopsied during the course of treatment (e.g., during the course of a clinical trial) and the new biopsy sample is used in a method of characterizing a disease described herein. Accordingly, this method involves determining the chromosomal instability metric in histological images from a patient receiving drug therapy, and correlating the levels with the cancer status of the patient (e.g., by comparison to predefined chromosomal instability metrics that correspond to different cancer statuses). The effect of therapy is determined based on these comparisons. If a treatment is effective, then the chromosomal instability metric of the histological images trend toward normal, while if treatment is ineffective, the chromosomal instability metric of the histological images trend toward cancer indications.

In some embodiments, the methods described herein are used to stratify patients with a disease (e.g., cancer) based on chromosomal instability for the clinical applications described herein. For example, the methods could be performed on a plurality of histological images from multiple patients and the resulting chromosomal instability metrics can be used to separate patients into different diagnostic or treatment groups.

### Exemplary Embodiments

The following embodiments are exemplary and are not intended to limit the scope of the invention described herein.

Embodiment 1. A method for characterizing a disease in a patient, comprising:
inputting one or more input histological images of a biological sample into a trained machine-learning model trained using a plurality of annotated training histological images;
identifying one or more mitotic events in the one or more input histological images using the trained machine-learning model, wherein the one or more mitotic events can be normal or abnormal mitotic events;
determining a frequency of the abnormal mitotic events in the one or more input histological images based on the identified mitotic events; and
classifying a pathological status of the one or more input histological images based on the determined frequency of abnormal mitotic events.

Embodiment 2. The method of embodiment 1, wherein the biological sample comprises at least a portion of a solid tumor.

Embodiment 3. The method of embodiment 2, wherein the at least a portion of the solid tumor is a biopsy slice of a solid tumor.

Embodiment 4. The method of any one of claims 1-3, wherein the biological sample relates to a plurality of training or input histological images from the same patient.

Embodiment 5. The method of any one of embodiments 1-4, wherein the one or more input histological images and/or the plurality of training histological images is an image captured at a resolution between 256 pixel×256 pixel and 10,000 pixel×10,000 pixel.

Embodiment 6. The method of any one of embodiments 1-5, wherein the one or more input histological images and/or the plurality of training histological images is captured between 20× and 100× magnification.

Embodiment 7. The method of any one of embodiments 1-6, the one or more input histological images and/or the plurality of training histological images are hematoxylin and eosin (H&E) stained images.

Embodiment 8. The method of any one of embodiments 1-7, further comprising segmenting one or more whole images into a plurality of image tiles, wherein the image tiles are inputted into the machine-learning model as the input histological images and/or the training histological images.

Embodiment 9. The method of any one of embodiments 1-7, wherein the machine-learning model segments the input histological images and/or the training histological images into tiles.

Embodiment 10. The method of any one of embodiments 1-9, wherein the input histological images and/or the training histological images are segmented by QuPath, U-Net, One Hundred Layers Tiramisu, or DenseNet.

Embodiment 11. The method of any one of embodiments 1-10, wherein the input histological images and/or the training histological images are processed to remove non-tumor tissue.

Embodiment 12. The method of any one of embodiments 1-11, wherein the annotated training histological images are annotated by an individual.

Embodiment 13. The method of any one of embodiments 1-11, wherein the annotated training histological images are annotated by a plurality of individuals.

Embodiment 14. The method of embodiment 13, further comprising selecting a set of high confidence annotated training histological images from the annotated training histological images.

Embodiment 15. The method of embodiment 14, wherein the set of high confidence annotated training histological images are selected based on concordance between annotations performed by a plurality of individuals.

Embodiment 16. The method of any one of embodiments 1-15, wherein the annotated training histological images include negative instances, wherein the negative instances are training histological images without a mitotic event.

Embodiment 17. The method of any one of embodiments 1-16, wherein one or more of the input histological images and/or the plurality of training images are deposited into computer cloud storage.

Embodiment 18. The method of any one of embodiments 1-17, wherein the machine-learning model is an unsupervised model.

Embodiment 19. The method of any one of embodiments 1-17, wherein the machine-learning model is a weakly-supervised model.

Embodiment 20. The method of any one of embodiments 1-17, wherein the machine-learning model is a human-in-the-loop model.

Embodiment 21. The method of any one of embodiments 1-20, wherein the machine-learning model applies a model selected from the group consisting of Support Vector Machines (SVM), Random Forests (RF), Artificial Neural Network (ANN), Convolutional Neural Network (CNN), K-means, ResNet, DenseNet, eXtreme Gradient Boosting (XGBoost), VGG, swin-transformer, Faster-RCNN, Mask-RCNN, and CentralNet++.

Embodiment 22. The method of any one of embodiments 1-21, wherein the machine-learning model uses a probability metric for a predicted classification of the mitotic event.

Embodiment 23. The method of any one of embodiments 1-22, wherein the annotated events with confidence above a specified threshold are used to compute a chromosomal instability metric.

Embodiment 24. The method of embodiment 23, wherein the chromosomal instability metric is the frequency of abnormal mitotic events with confidence above a specified threshold compared to all mitotic events in an input histological image.

Embodiment 25. The method of embodiment 23, wherein the chromosomal instability metric is the frequency of abnormal mitotic events with confidence above a specified threshold compared to all mitotic events in an input histological image tile.

Embodiment 26. The method of embodiment 23, wherein the chromosomal instability metric is the frequency of abnormal mitotic events with confidence above a specified threshold compared to all mitotic events in an input histological image tile normalized by a tumor area metric, wherein the tumor area metric is the tumor area calculated using a tumor image segmentor.

Embodiment 27. The method of any one of embodiments 1-26, wherein the machine-learning model outputs a chromosome instability metric for each input histological image tile.

Embodiment 28. The method of any one of embodiments 1-26, wherein the machine-learning model outputs a chromosome instability metric aggregated for all input histological image tiles.

Embodiment 29. The method of any one of embodiments 23-28, wherein the chromosomal instability metric is related to the pathological status of the input histological images.

Embodiment 30. The method of any one of embodiments 23-29, wherein the chromosomal instability metric is displayed to a user.

Embodiment 31. The method of any one of embodiments 1-30, wherein characterizing a disease comprises diagnosing the disease.

Embodiment 32. The method of any one of embodiments 1-30, wherein characterizing a disease comprises informing a treatment strategy.

Embodiment 33. The method of any one of embodiments 1-30, wherein characterizing a disease comprises evaluating the disease progression.

Embodiment 34. The method of any one of embodiments 1-30, wherein characterizing a disease comprises predicting the disease prognosis.

Embodiment 35. The method of any one of embodiments 1-30, wherein characterizing a disease comprises evaluating effect of a treatment

Embodiment 36. The method of any one of embodiments 1-30, wherein characterizing a disease comprises identifying a patient population for treatment.

Embodiment 37. The method of any one of embodiments 1-36, wherein the disease is a cancer.

Embodiment 38. The method of any one of embodiments 1-37, wherein the method is implemented on a cloud-based computing platform.

Embodiment 39. A system for characterizing a disease in a patient with machine-learning, comprising: one or more processors; a memory; and one or more programs with instructions for:
receiving data representing one or more input histological images of a biological sample;
identifying one or more mitotic events in the one or more input histological images using a trained machine-learning model trained using a plurality of annotated training histological images, wherein the one or more mitotic events can be normal or abnormal mitotic events;
determining a frequency of the abnormal mitotic events in the one or more input histological images based on the identified mitotic events; and
classifying a pathological status of the one or more input histological images based on the determined frequency of abnormal mitotic events.

Embodiment 40. The system of embodiment 39, wherein the biological sample comprises at least a portion of a solid tumor.

Embodiment 41. The system of embodiment 40, wherein the at least a portion of the solid tumor is a biopsy slice of a solid tumor.

Embodiment 42. The system of any one of embodiments 39-41, wherein the biological sample relates to a plurality of training or input histological images from the same patient.

Embodiment 43. The system of any one of embodiments 39-41, wherein one or more input histological images and/or the plurality of training histological images is an image captured at a resolution between 256 pixel×256 pixel and 10,000 pixel×10,000 pixel.

Embodiment 44. The system of any one of embodiments 39-43, wherein one or more of the input histological images and/or the plurality of training histological images is captured at between 20× and 100× magnification.

Embodiment 45. The system of any one of embodiments 39-44, wherein one or more of the input histological images and/or the plurality of training histological images are hematoxylin and eosin (H&E) stained images.

Embodiment 46. The system of any one of embodiments 39-45, wherein the instructions further comprise instructions for segmenting one or more whole images into a plurality of image tiles, wherein the image tiles are inputted into the machine-learning model as the input histological images and/or the training histological images.

Embodiment 47. The system of any one of embodiments 39-46, wherein the machine-learning model segments the input histological images and/or the training histological images into tiles.

Embodiment 48. The system of any one of embodiments 39-47, wherein the input histological images and/or the training histological images are segmented by QuPath, U-Net, One Hundred Layers Tiramisu, or DenseNet.

Embodiment 49. The system of any one of embodiments 39-48, wherein the input histological images and/or the training histological images are processed to remove non-tumor tissue.

Embodiment 50. The system of any one of embodiments 39-49, wherein the annotated training histological images are annotated by an individual.

Embodiment 51. The system of any one of embodiments 39-49, wherein the annotated training histological images are annotated by a plurality of individuals.

Embodiment 52. The system of embodiment 51, wherein a set of high confidence annotated training histological images are selected from the annotated training histological images.

Embodiment 53. The system of embodiment 52, wherein the set of high confidence annotated training histological images are selected based on concordance between annotations performed by a plurality of individuals.

Embodiment 54. The system of any one of embodiments 39-53, wherein the annotated training histological images include negative instances, wherein the negative instances are training histological images without a mitotic event.

Embodiment 55. The system of any one of embodiments 39-54, wherein one or more of the input histological image and/or the plurality of training histological images are deposited into a computer cloud.

Embodiment 56. The system of any one of embodiments 39-55, wherein the machine-learning model is an unsupervised model.

Embodiment 57. The system of any one of embodiments 39-55, wherein the machine-learning model is a weakly-supervised model.

Embodiment 58. The system of any one of embodiments 39-55, wherein the machine-learning model is a human-in-the-loop model.

Embodiment 59. The system of any one of embodiments 39-58, wherein the machine-learning model applies a model selected from the group consisting of Support Vector Machines (SVM), Random Forests (RF), Artificial Neural Network (ANN), Convolutional Neural Network (CNN), K-means, ResNet, DenseNet, eXtreme Gradient Boosting (XGBoost), VGG, swin-transformer, Faster-RCNN, Mask-RCNN, and CentralNet++.

Embodiment 60. The system of any one of embodiments 39-59, wherein the machine-learning model uses a uses a probability metric for a predicted classification of the mitotic event and/or wherein the annotated events with confidence above a specified threshold are used to compute a chromosomal instability metric.

Embodiment 61. The system of claim 60, wherein the chromosomal instability metric is the frequency of abnormal mitotic events with confidence above a specified threshold compared to all mitotic events in an input histological image.

Embodiment 62. The system of embodiment 60, wherein the chromosomal instability metric is the frequency of abnormal mitotic events with confidence above a specified threshold compared to all mitotic events in an input histological image tile.

Embodiment 63. The system of embodiment 60, wherein the chromosomal instability metric is the frequency of abnormal mitotic events with confidence above a specified threshold compared to all mitotic events in an input histological image tile normalized by a tumor area metric.

Embodiment 64. The system of embodiment 63, wherein the tumor area metric is the tumor area calculated using a tumor image segmentor.

Embodiment 65. The system of any one of embodiments 39-64, wherein the machine-learning model outputs a chromosome instability metric for each input histological image tile.

Embodiment 66. The system of any one of embodiments 39-64, wherein the machine-learning model outputs a chromosome instability metric aggregated for all input histological image tiles.

Embodiment 67. The system of any one of embodiments 60-66, wherein the chromosomal instability metric is related to the pathological status of the input histological image.

Embodiment 68. The system of any one of embodiments 60-67, wherein the chromosomal instability metric is displayed to a user.

Embodiment 69. The system of any one of embodiments 39-68, wherein characterizing a disease comprises diagnosing the disease.

Embodiment 70. The system of any one of embodiments 39-68, wherein characterizing a disease comprises informing a treatment strategy.

Embodiment 71. The system of any one of embodiments 39-68, wherein characterizing a disease comprises evaluating the disease progression.

Embodiment 72. The system of any one of embodiments 39-68, wherein characterizing a disease comprises predicting the disease prognosis.

Embodiment 73. The system of any one of embodiments 39-68, wherein characterizing a disease comprises evaluating effect of a treatment.

Embodiment 74. The system of any one of embodiments 39-68, wherein characterizing a disease comprises identifying a patient population for treatment.

Embodiment 75. The system of any one of embodiments 39-74, wherein the disease is a cancer.

Embodiment 76. The system of any one of embodiments 39-75, wherein the instructions are implemented on a cloud-based computing platform.

Embodiment The system of any one of embodiments 39-76, wherein the instructions for implementing the instructions reside in cloud storage.

Embodiment 78. A method for training a machine-learning model to analyze histological images of biological samples, comprising:
annotating the plurality of training histological images by identifying both normal and abnormal mitotic events in the plurality of training histological images; and
training the machine-learning model based on the annotated histological images, wherein the machine-learning model is configured to receive one or more input histological images and determine a pathological metric of the one or more input histological images.

Embodiment 79. The method of embodiment 78, wherein the biological sample comprises at least a portion of a solid tumor.

Embodiment 80. The method of embodiment 79, wherein the at least a portion of the solid tumor is a biopsy slice of a tumor.

Embodiment 81. The method of any one of embodiments 78-80, wherein the biological sample relates to a plurality of training or input histological images from the same patient.

Embodiment 82. The method of any one of embodiments 78-81, wherein the one or more input histological images and/or the plurality of training histological images is an image captured at a resolution between 256 pixel×256 pixel and 10,000 pixel×10,000 pixel resolution.

Embodiment 83. The method of any one of embodiments 78-82, wherein the one or more input histological images and/or the plurality of training histological images is captured at between 20× and 100× magnification.

Embodiment 84. The method of any one of embodiments 78-83, wherein the one or more input histological images and/or the plurality of training histological images are hematoxylin and eosin (H&E) stained images.

Embodiment 85. The method of any one of embodiments 78-84, further comprising segmenting one or more whole images into a plurality of image tiles, wherein the image tiles are inputted into the machine-learning model as the input histological images and/or the training histological images.

Embodiment 86. The method of any one of embodiments 78-85, wherein the machine-learning model segments the input histological images and/or the training histological images into tiles.

Embodiment 87. The method of any one of embodiments 78-86, wherein the input histological images and/or the training histological images are segmented by QuPath, U-Net, One Hundred Layers Tiramisu, or DenseNet.

Embodiment 88. The method of any one of embodiments 78-87, wherein the input histological images and/or the training histological images are processed to remove non-tumor tissue.

Embodiment 89. The method of any one of embodiments 78-88, wherein the annotated training histological images are annotated by an individual.

Embodiment 90. The method of embodiment 89, wherein the individual is a person.

Embodiment 91. The method of embodiment 90, wherein the person is a pathologist, a biologist, and/or a medical professional.

Embodiment 92. The method of any one of embodiments 78-91, wherein the annotating comprises identifying both normal and abnormal mitotic events in the training histological images.

Embodiment 93. The method of any one of embodiments 78-92, wherein the annotating comprises tracing the boundaries of both normal and abnormal mitotic events in the training histological images.

Embodiment 94. The method of any one of embodiments 78-93, wherein the annotating comprises classifying the mitotic events as normal or abnormal.

Embodiment 95. The method of any one of embodiments 78-94, wherein the annotating comprises classifying the normal and abnormal mitotic events with a level of confidence the mitotic event is normal or abnormal, respectively.

Embodiment 96. The method of any one of embodiments 78-91, wherein at least one of the abnormal mitotic event is selected from the group consisting of unaligned metaphase, multipolar pre-anaphase, lagging chromosomes in anaphase, chromosomal bridges in anaphase or telophase, mixed lagging chromosome and chromosomal bridges in anaphase or telophase, and multipolar anaphase or telophase.

Embodiment 97. The method of any one of embodiments 92-96, wherein at least one of the normal mitotic event is selected from the group consisting of prometaphase, normal metaphase, and normal anaphase.

Embodiment 98. The method of any one of embodiments 78-97, wherein the annotated training histological images are annotated by a plurality of individuals.

Embodiment 99. The method of any one of embodiments 78-98, further comprising selecting a set of high confidence annotated training histological images from the annotated training histological images.

Embodiment 100. The method of embodiment 99, wherein the set of high confidence annotated training histological images are selected based on concordance between annotations performed by a plurality of individuals.

Embodiment 101. The method of any one of embodiments 78-100, wherein the annotated training histological images include negative instances, wherein the negative instances are training histological images without a mitotic event.

Embodiment 102. The method of any one of embodiments 78-101, wherein one or more of the input histological image and/or the plurality of training histological images are deposited into computer cloud storage.

Embodiment 103. The method any one of embodiments 78-102, wherein the machine-learning model is not trained using a genomic score.

Embodiment 104. The method of any one of embodiments 78-103, wherein the machine-learning model is an unsupervised model.

Embodiment 105. The method of any one of embodiments 78-103, wherein the machine-learning model is a weakly-supervised model.

Embodiment 106. The method of any one of embodiments 78-103, wherein the machine-learning model is a human-in-the-loop model.

Embodiment 107. The method of any one of embodiments 78-106, wherein the machine-learning model applies a model selected from the group consisting of Support Vector Machines (SVM), Random Forests (RF), Artificial Neural Network (ANN), Convolutional Neural Network (CNN), K-means, ResNet, DenseNet, eXtreme Gradient Boosting (XGBoost), VGG, swin-transformer, Faster-RCNN, Mask-RCNN, and CentralNet++.

Embodiment 108. The method of any one of embodiments 78-107, the machine-learning model predicts a pathological metric for each input histological image.

Embodiment 109. The method of embodiment 108, wherein the pathological metric is a chromosomal instability metric.

Embodiment 110. The method of embodiment 109, wherein the chromosomal instability metric is related to the pathological status of the input histological image.

Embodiment 111. The method of embodiment 110, wherein the chromosomal instability metric is the frequency of abnormal mitotic events compared to all mitotic events in an inputted histological image.

Embodiment 112. The method of embodiment 110, wherein the chromosomal instability metric is the frequency of abnormal mitotic events compared to all mitotic events in an input histological image tile.

Embodiment 113. The method of any one of embodiments 78-112, wherein the machine-learning model outputs a chromosome instability metric for each input histological image tile.

Embodiment 114. The method of any one of embodiments 78-112, wherein the machine-learning model outputs a chromosome instability metric aggregated for all input histological image tiles.

Embodiment 115. The method of any one of embodiments 109-114, wherein the chromosomal instability metric is the number of abnormal mitotic events normalized by tumor size.

Embodiment 116. The method of any one of embodiments 109-115, wherein the chromosomal instability metric is the frequency of abnormal mitotic events with confidence above a specified threshold compared to all mitotic events in an input histological image tile normalized by a tumor area metric.

Embodiment 117. The method of embodiment 116, wherein the tumor area metric is the tumor area calculated using a tumor image segmentor.

Embodiment 118. The method of any one of embodiments 109-117, wherein the chromosomal instability metric is displayed to a user.

Embodiment 119. The method of any one of embodiments 78-118, wherein the method is implemented on a cloud-based computing platform.

Embodiment 120. A system for training a machine-learning model to analyze histological images of biological samples comprising one or more processors, memory, and one or more applications stored in the memory that include instructions for:
receiving a plurality of annotated training histological images by identifying both normal and abnormal mitotic events in the plurality of training histological images; and
training the machine-learning model based on the annotated histological images, wherein the machine-learning model is configured to receive one or more input histological images and determine a pathological metric of the one or more input histological images.

Embodiment 121. The system of embodiment 120, wherein the biological sample comprises at least a portion of a solid tumor.

Embodiment 122. The system of embodiment 121, wherein the at least a portion of the solid tumor is a biopsy slice of a solid tumor.

Embodiment 123. The system of any one of embodiments 120-122, wherein the biological sample relates to a plurality of training or input histological images from the same patient.

Embodiment 124. The system of any one of embodiments 120-123, wherein the one or more input histological images and/or the plurality of histological images is an image captured at a resolution between 256 pixel×256 pixel and 10,000 pixel×10,000 pixel.

Embodiment 125. The system of any one of embodiments 120-124, wherein the one or more input histological images and/or the plurality of training histological images is captured at between 20× and 100× magnification.

Embodiment 126. The system of any one of embodiments 120-125, wherein the one or more input histological images and/or the plurality of training histological images are hematoxylin and eosin (H&E) stained images.

Embodiment 127. The system of any one of embodiments 120-126, wherein the instructions further comprise instructions for segmenting one or more whole images into a plurality of image tiles, wherein the image tiles are inputted into the machine-learning model as the input histological images and/or the training histological images.

Embodiment 128. The system of any one of embodiments 120-127, wherein the machine-learning model segments the input histological images and/or the training histological images into tiles.

Embodiment 129. The system of any one of embodiments 120-128, wherein the input histological images and/or the training histological images are segmented by QuPath, U-Net, One Hundred Layers Tiramisu, or DenseNet.

Embodiment 130. The system of any one of embodiments 120-129, wherein the input histological images and/or the training histological images are processed to remove non-tumor tissue.

Embodiment 131. The system of any one of embodiments 120-130, wherein the annotated training histological images are annotated by an individual.

Embodiment 132. The system of embodiment 131, wherein the individual is a person.

Embodiment 133. The system of embodiment 132, wherein the person is a pathologist, a biologist, and/or a medical professional.

Embodiment 134. The system of any one of embodiments 120-130, wherein the annotated training histological images are annotated by a plurality of individuals.

Embodiment 135. The system of embodiment 134, wherein a set of high confidence annotated training histological images are selected from the annotated training histological images.

Embodiment 136. The system of embodiment 135, wherein the set of high confidence annotated training histological images are selected based on concordance between annotations performed by a plurality of individuals.

Embodiment 137. The system of any one of embodiments 120-136, wherein the annotated training histological images include negative instances, wherein the negative instances are training histological images without a mitotic event.

Embodiment 138. The system of any one of embodiments 131-137, wherein the annotating comprises identifying both normal and abnormal mitotic events in the training histological images.

Embodiment 139. The system of any one of embodiments 131-138, wherein the annotating comprises tracing the boundaries of both normal and abnormal mitotic events in the training histological images.

Embodiment 140. The system of any one of embodiments 131-139, wherein the annotating comprises classifying the mitotic events as normal or abnormal.

Embodiment 141. The system of embodiment 140, wherein the annotation includes classifying the normal and abnormal mitotic events with a level of confidence the mitotic event is normal or abnormal, respectively.

Embodiment 142. The system of any one of embodiments 138-141, wherein at least one of the abnormal mitotic event is selected from the group consisting of unaligned metaphase, multipolar pre-anaphase, lagging chromosomes in anaphase or telophase, chromosomal bridges in anaphase or telophase, mixed lagging chromosome and chromosomal bridges in anaphase or telophase, and multipolar anaphase or telophase.

Embodiment 143. The system of any one of embodiments 138-107, wherein at least one of the normal mitotic event is selected from the group consisting of prometaphase, normal metaphase, and normal anaphase.

Embodiment 144. The system of any one of embodiments 120-143, wherein the one or more input histological images and/or the plurality of training histological images are deposited into computer cloud storage.

Embodiment 145. The system of any one of embodiments 120-144, wherein the machine-learning model is not trained using a genomic score.

Embodiment 146. The system of any one of embodiments 120-145, wherein the machine-learning model is an unsupervised model.

Embodiment 147. The system of any one of embodiments 120-145, wherein the machine-learning model is a weakly-supervised model.

Embodiment 148. The system of any one of embodiments 120-145, wherein the machine-learning model is a human-in-the-loop model.

Embodiment 149. The system of any one of embodiments 120-148, wherein the machine-learning model applies a model selected from the group consisting of Support Vector Machines (SVM), Random Forests (RF), Artificial Neural Network (ANN), Convolutional Neural Network (CNN), K-means, ResNet, DenseNet, eXtreme Gradient Boosting (XGBoost), VGG, swin-transformer, Faster-RCNN, Mask-RCNN, and CentralNet++.

Embodiment 150. The system of any one of embodiments 120-149, wherein the machine-learning model predicts a pathological metric for each input histological image.

Embodiment 151. The system of embodiment 150, where in the pathological metric is a chromosomal instability metric.

Embodiment 152. The system of embodiment 151, wherein the chromosomal instability metric is related to the pathological status of the input histological image.

Embodiment 153. The system of embodiment 152, wherein the chromosomal instability metric is the frequency of abnormal mitotic events compared to all mitotic events in an input histological image.

Embodiment 154. The system of embodiment 152, wherein the chromosomal instability metric is the frequency of abnormal mitotic events compared to all mitotic events in an input histological image tile.

Embodiment 155. The system of any one of embodiments 120-154, wherein the machine-learning model outputs a chromosome instability metric for each input histological image tile.

Embodiment 156. The system of any one of embodiments 120-154, wherein the machine-learning model outputs a chromosome instability metric aggregated for all input histological image tiles.

Embodiment 157. The system of any one of embodiments 151-156, wherein the chromosomal instability metric is the number of abnormal mitotic events normalized by tumor size.

Embodiment 158. The system of any one of embodiments 151-157, wherein the chromosomal instability metric is the frequency of abnormal mitotic events with confidence above a specified threshold compared to all mitotic events in an input histological image tile normalized by a tumor area metric.

Embodiment 159. The system of embodiment 158, wherein the tumor area metric is the tumor area calculated using an image segmentor.

Embodiment 160. The system of any one of embodiments 151-159, wherein the chromosomal instability metric is displayed to a user.

Embodiment 161. The system of any one of embodiments 120-160, wherein the instructions are implemented on a cloud-based computing platform

Embodiment 162. The system of any one of embodiments 120-161, wherein the instructions for implementing the instructions reside in cloud storage.

Embodiment 163. A non-transitory computer-readable storage medium storing one or more programs, the one or more programs comprising instructions, which when executed by one or more processors of an electronic device having a display, which when executed by an electric device cause the electronic device to:
receive one or more input histological images of a biological sample;
identify one or more mitotic events in the one or more input histological images using a trained machine-learning model trained using a plurality of annotated training histological images, wherein the one or more mitotic events can be normal or abnormal mitotic events;
determine a frequency of the abnormal mitotic events in the one or more input histological images based on the identified mitotic events; and
classify a pathological status of the one or more input histological images based on the determined frequency of abnormal mitotic events.

Embodiment 164. The computer-readable storage medium of embodiment 163, wherein the biological sample comprises at least a portion of a solid tumor.

Embodiment 165. The computer-readable storage medium of embodiment 164, wherein the at least a portion of the solid tumor is a biopsy slice of a tumor.

Embodiment 166. The computer-readable storage medium of any one of embodiments 163-165, wherein the biological sample relates to a plurality of training or input histological images from the same patient.

Embodiment 167. The computer-readable storage medium of any one of embodiments 163-166, wherein the one or more input histological images and/or the plurality of training histological images is an images captured at a resolution between 256 pixel×256 pixel and 10,000 pixel×10,000 pixel.

Embodiment 168. The computer-readable storage medium of any one of embodiments 163-167, wherein the one or more input histological images and/or the plurality of training histological images is captured at between 20× and 100× magnification.

Embodiment 169. The computer-readable storage medium of any one of embodiments 163-168, wherein the one or more input histological images and/or the plurality training histological images are hematoxylin and eosin (H&E) stained images.

Embodiment 170. The computer-readable storage medium of any one of embodiments 163-169, wherein the instructions further comprise instructions for segmenting one or more whole images into a plurality of image tiles, wherein the image tiles are inputted into the machine-learning model as the input histological images and/or the training histological images.

Embodiment 171. The computer-readable storage medium of any one of embodiments 163-170, wherein the machine-learning model segments the input histological images and/or the training histological images into tiles.

Embodiment 172. The computer-readable storage medium of any one of embodiments 163-171, wherein the input histological images and/or the training histological images are segmented by QuPath, U-Net, One Hundred Layers Tiramisu, or DenseNet.

Embodiment 173. The computer-readable storage medium of any one of embodiments 163-172, wherein the input histological images and/or the training histological images are processed to remove non-tumor tissue.

Embodiment 174. The computer-readable storage medium of any one of embodiments 163-173, wherein the annotated training histological images are annotated by an individual.

Embodiment 175. The computer-readable storage medium of embodiment 174, wherein the individual is a person.

Embodiment 176. The computer-readable storage medium of embodiment 175, wherein the person is a pathologist, a biologist, and/or a medical professional.

Embodiment 177. The computer-readable storage medium of any one of embodiments 174-176, wherein the annotating comprises identifying both normal and abnormal mitotic events in the training histological images.

Embodiment 178. The computer-readable storage medium of any one of embodiments 174-177, wherein the annotating comprises tracing the boundaries of both normal and abnormal mitotic events in the training histological images.

Embodiment 179. The computer-readable storage medium of any one of embodiments 174-178, wherein the annotating comprises classifying the mitotic events as normal or abnormal.

Embodiment 180. The computer-readable storage medium of any one of embodiments 174-179, wherein the annotating comprises classifying the normal and abnormal mitotic events with a level of confidence the mitotic event is normal or abnormal, respectively.

Embodiment 181. The computer-readable storage medium of any one of embodiments 177-180, wherein at least one of the abnormal mitotic event is selected from the group consisting of unaligned metaphase, multipolar pre-anaphase, lagging chromosomes in anaphase or telophase, chromosomal bridges in anaphase or telophase, mixed lagging chromosome and chromosomal bridges in anaphase or telophase, and multipolar anaphase or telophase.

Embodiment 182. The computer-readable storage medium of any one of embodiments 177-181, wherein at least one of the normal mitotic event is selected from the group consisting of prometaphase, normal metaphase, and normal anaphase.

Embodiment 183. The computer-readable storage medium of any one of embodiments 174-182, wherein the annotated training histological images are annotated by a plurality of individuals.

Embodiment 184. The computer-readable storage medium embodiment 183, wherein the annotated training histological images are a set of high confidence annotated training histological images selected from the annotated training histological images.

Embodiment 185. The computer-readable storage medium of embodiment 184, wherein the set of high confidence annotated training histological images are selected based on concordance between annotations performed by a plurality of individuals.

Embodiment 186. The computer-readable storage medium of any one of embodiments 163-185, wherein the annotated training histological images include negative instances, wherein the negative instances are training histological images without a mitotic event.

Embodiment 187. The computer-readable storage medium of any one of embodiments 163-187, wherein the machine-learning model is not trained using a genomic score.

Embodiment 188. The computer-readable storage medium of any one of embodiments 163-187, wherein the machine-learning model is an unsupervised model.

Embodiment 189. The computer-readable storage medium of any one of embodiments 163-187, wherein the machine-learning model is a weakly-supervised model.

Embodiment 190. The computer-readable storage medium of any one of embodiments 163-187, wherein the machine-learning model is a human-in-the-loop model.

Embodiment 191. The computer-readable storage medium of any one of embodiments 163-190, wherein the machine-learning model applies a model selected from the group consisting of Support Vector Machines (SVM), Random Forests (RF), Artificial Neural Net (ANN), Convolutional Neural Net (CNN), K-means, ResNet, DenseNet, eXtreme Gradient Boosting (XGBoost), VGG, swin-transformer, Faster-RCNN, Mask-RCNN, and CentralNet++.

Embodiment 192. The computer-readable storage medium of any one of embodiments 163-191, wherein the machine-learning model predicts a pathological metric for each input histological image.

Embodiment 193. The computer-readable storage medium of embodiment 192, where in the pathological metric is a chromosomal instability metric.

Embodiment 194. The computer-readable storage medium of embodiment 193, wherein the chromosomal instability metric is related to the pathological status of the input histological image.

Embodiment 195. The computer-readable storage medium of any one of embodiments 163-194, wherein the machine-learning model uses a uses a probability metric for a predicted classification of the mitotic event.

Embodiment 196. The computer-readable storage medium of embodiment 194, wherein the chromosomal instability metric is the frequency of abnormal mitotic events with confidence above a specified threshold compared to all mitotic events in an input histological image.

Embodiment 197. The computer-readable storage medium of embodiment 194, wherein the chromosomal instability metric is the frequency of abnormal mitotic events with confidence above a specified threshold compared to all mitotic events in an input histological image tile.

Embodiment 198. The computer-readable storage medium of embodiments 163-197, wherein the machine-learning model outputs a chromosome instability metric for each input histological image tile.

Embodiment 199. The computer-readable storage medium of embodiments 163-198, wherein the machine-learning model outputs a chromosome instability metric aggregated for all input histological image tiles.

Embodiment 200. The computer-readable storage medium of any one of embodiments 193-199, wherein the chromosomal instability pathological metric is the number of abnormal mitotic events normalized by tumor size.

Embodiment 201. The computer-readable storage medium of any one of embodiments 193-200, wherein the chromosomal instability metric is the frequency of abnormal mitotic events with confidence above a specified threshold compared to all mitotic events in an input histological image tile normalized by a tumor area metric.

Embodiment 202. The computer-readable storage medium of embodiment 201, wherein the tumor area metric is the tumor area calculated using a tumor image segmentor.

Embodiment 203. The computer-readable storage medium of any one of embodiments 193-202, wherein the chromosomal instability metric is displayed to a user.

Embodiment 204. The computer-readable storage medium of any one of embodiments 163-203, wherein the one or more computer programs are implemented on a cloud-based computing platform.

Embodiment 205. The computer-readable storage medium of any one of embodiments 163-204, wherein the instructions for implementing the one or more computer programs reside in cloud storage.

### EXAMPLES

The examples below are intended to be purely exemplary of the invention and should therefore not be considered to limit the invention in any way. The following examples and detailed description are offered by way of illustration and not by way of limitation.

### Example 1: Training a Machine-Learning Model Using Biological Images from the Cancer Genome Atlas (TCGA)

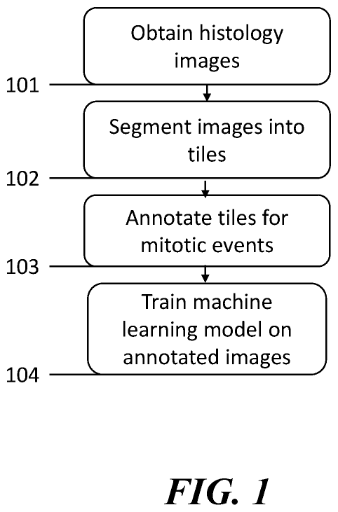

This example demonstrates a method for training a machine-learning model that can detect and quantify abnormal mitotic events and calculate a chromosomal instability (CIN) score from stained biological images obtained from a biological sample (FIG. 1). In particular, this example includes training a convolutional neural network (CNN) model with hematoxylin and eosin (H&E) stained training histological images from the TCGA database that have been annotated for normal and abnormal mitotic events. This exemplary method outputs a model prepared to receive input histological images from patient tumor samples and output annotated images. The model can also output a frequency of abnormal mitotic events in the form of a CIN score, which can inform cancer treatment.

The machine-learning model is trained using training histological images. Training images are selected and downloaded from the TCGA database (101). Each image is an H&E stained histopathology image of a patient tumor. In some embodiments, the tumor may be a primary tumor or a metastatic tumor. The tumor type that may be used for the training methods described herein is not limited, such that the method is applicable to a wide variety of cancer types and cancer stages. Selected histological training images may vary in their degree of CIN, which may be evaluated by a variety of CIN metrics including but not limited to the frequency of abnormal mitotic events in the training histological images. The histological training images may be at a magnification that allows for the specific detection of mitotic events, such as 40× magnification. Patient training histological images may be segmented into tiles (e.g., portions of the whole training histological image). For example, the training histological images may be segmented into 1,024 pixels×1,024 pixels tiles (102).

The training images may be preprocessed with a tumor segmentor. The tumor segmentor may remove non-tumor area in the image such as stroma or fat. FIG. 8. The tumor segmentor may be semi-automatic using QuPath and expert knowledge from a pathologist. The tumor segmentor may also be an automatic method using an algorithm such as DenseNet.

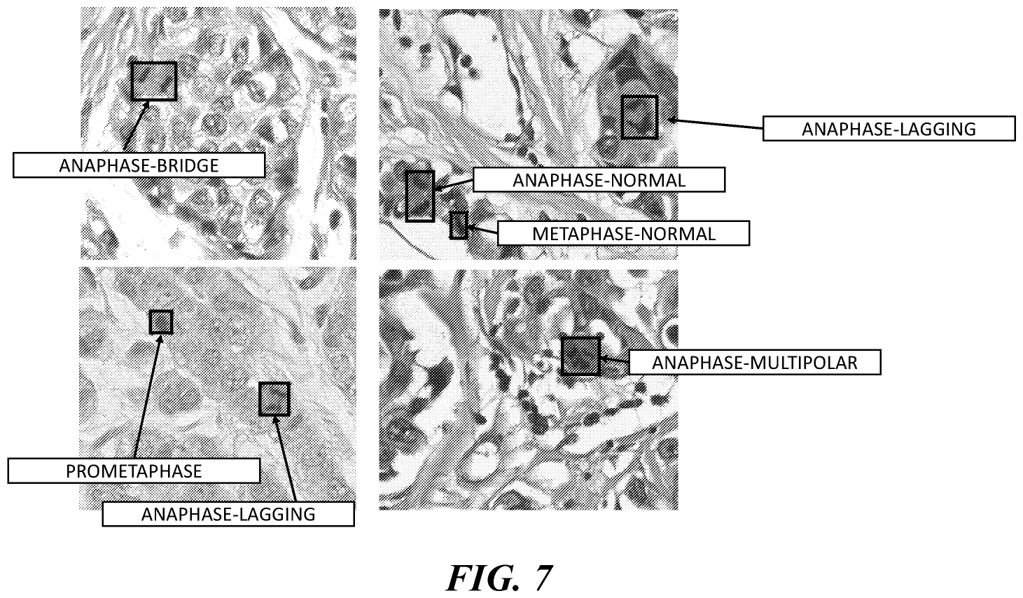

Each training histological image, or tile thereof, is annotated by labeling normal and abnormal mitotic events. In some embodiments, the training histological images or training histological image tiles thereof are annotated by any professional capable of identifying mitotic events in a stained image of a biological sample. Abnormal mitotic events may include, but are not limited to, unaligned metaphase, multipolar pre-anaphase, lagging chromosomes in anaphase or telophase, chromosomal bridges in anaphase or telophase, mixed lagging chromosome and chromosomal bridges in anaphase or telophase, and multipolar anaphase or telophase. (103). The abnormal events may result in daughter cells with gains or losses of full chromosomes or amplifications, deletions, inversions, or translocations of chromosomal regions, ranging from a single gene to a full chromosome arms. Normal mitotic events may include prometaphase, normal metaphase, and normal anaphase. FIG. 7 provides non-limiting examples of histological images and the corresponding labels annotating abnormal and normal mitotic events in those images. In FIG. 7, annotated normal mitotic events include prometaphase and normal anaphase, and annotated abnormal mitotic events include unaligned metaphase, multipolar pre-anaphase, lagging chromosomes in anaphase or telophase, polar chromosomes (those located at the spindle poles) in metaphase or anaphase or telophase, chromosomal bridges in anaphase or telophase, mixed lagging chromosome and chromosomal bridges in anaphase or telophase, multipolar anaphase or telophase, or any combination thereof. Images are captured at 40× magnification and varying levels of resolution.

Each training histological may be annotated by first identifying mitotic events, followed by further classifying the identified mitotic events. The mitotic event annotation may be crowd-sourced, where an individual or a plurality of individuals identify (e.g., annotate by outline) individual mitotic events in histological images. The outlined mitotic events may be further characterized as normal or abnormal by an individual or a plurality of individuals. The classification maybe include confidence levels, such as, “certain” normal anaphase, “likely” normal anaphase, “certain” abnormal anaphase, “likely abnormal anaphase”, or non-anaphase.

The annotated training images may be annotated by a plurality of individuals. Additionally, the annotated training images may be annotated using various annotation methods. The concordance between annotations performed by each individual of a plurality of individuals may be determined.

The annotated training images are deposited into a cloud-computing server, such as but not limited to, Microsoft Azure. A CNN machine-learning model is trained using the annotated training histological image tiles (104).

A variety of machine-learning, image processing, and data processing platforms may be used to implement the disclosed CIN detection methods described herein. Such methods may include Amazon Web Services (AWS) cloud computing services (e.g., using the P2 graphics processing unit (GPU) architecture), TensorFlow (an open-source program library for machine-learning), Apache Spark (an open-source, general-purpose distributed computing system used for big data analytics), Databricks (a web-based platform for working with Spark, that provides automated cluster management), Horovod (an open-source framework for distributed deep learning training using TensorFlow, Keras, PyTorch, and Apache MXNet), OpenSlide (a C library that provides an interface for reading whole-slide images), Scikit-Image (a collection of image processing methods), and Pyvips (a Python-based binding for the libvips image processing library).

### Example 2: Classifying and Quantifying CIN Events in Patient Primary Tumor Images

This example shows the deployment of the machine-learning model trained in Example 1 to classify and quantify CIN in a patient derived sample in a clinical setting (e.g., patient derived H&E stained input histological image of a biological sample). This example further illustrates using the model output to select a treatment based on the CIN status of the sample (FIG. 3 and FIG. 16).

Biological samples are prepared from patient tumor resections, such as biopsies. Biological samples are stained with H&E and fixed to a slide for histological imaging. Whole slide input histological images are captured at 40× magnification. Patient input histological images may be segmented into tiles. For example, the images may segmented into 1,024 pixels×1,024 pixels tiles (301). The input histological image tiles are inputted into the machine-learning model trained using annotated training histological images, as described in Example 1.

The machine-learning model is deployed to classify the mitotic events (e.g., normal mitotic events versus abnormal mitotic events, as well as type of abnormal mitotic event) in each input histological image tile according to the events in the training histological images and/or training histological image tiles (302). The machine-learning model assigns a quality score to each input histological image, or tile thereof, that is representative of how likely the input histological image is to contain the mitotic event of a specific class. Using the events identified in all of the input histological image tiles belonging to a whole input histological image, the machine-learning model outputs a metric corresponding to the number abnormal mitotic events normalized by the number of normal and abnormal mitotic events identified in all of the tiles belonging to the same whole input histological image (303).

The CIN metric may be used (e.g., by a medical professional) to classify a patient tumor according to the level of CIN detected. If a tumor is classified as having a high CIN metric, the physician may treat the patient with an anti-CIN cancer agent as described herein (304). The CIN metric may also be used for additional clinical applications such as to evaluate the effect of a treatment or diagnose a tumor stage.

The application may be better understood by reference to the following non-limiting examples, which are provided as exemplary embodiments of the application. The following examples are presented in order to more fully illustrate embodiments and should in no way be construed, however, as limiting the broad scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 3: Using a Pre-Clinical Mice H&E Images to Train a Deep-Learning System to Classify Normal and Abnormal Anaphase Figures for Measuring CIN

This example describes the training and use of a machine-learning model provided herein, using labeled anaphases events as training data obtained from mice and annotations from two experts for classifying normal anaphases from abnormal required to calculate the CIN.

The pre-clinical mice data was used to address the issue of lacking enough anaphase events in human cancer H&E images for training a deep learning model. 119 WSIs of 12 different mouse syngeneic primary tumor models were used for training. Anaphase events previously detected were classified by two experts.

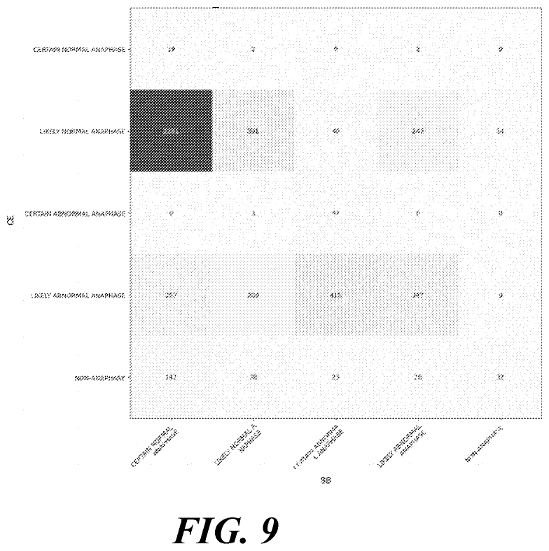

Whole slide images of mouse tumors were obtained and annotated by two individual expert pathologists. From 69 whole slide images (of 119 total whole slide images), 4,544 mitotic events, e.g., possible anaphase figures, were annotated by two individual experts. The experts classified each event as one of five categories: i) “certain” normal anaphase, ii) “likely” normal anaphase, iii) “certain” abnormal anaphase, iv) “likely” abnormal anaphase, or v) non-anaphase. FIG. 9 compares the annotations from each of the two experts for the 4,544 mitotic events. After aggregation of the annotations, there were 2770 total anaphase figures where both experts agreed on the directionality (e.g., normal anaphase or abnormal anaphase) and at least one expert was “certain” about the classification. 2.302 anaphase figures (out of 2,770) were classified as normal and 468 anaphase figures were classified as abnormal.

Data augmentation was used to balance the normal and abnormal images (2302 normal vs. 468 abnormal) obtained from section. The data augmentation strategies including rotated, distorted, and zooming-in were applied to the abnormal anaphase events to increase the number of abnormal events. After augmentation by 4 times, the abnormal anaphase figures were increased to 2,340. Subsequently, the number of all training data including both normal and augmented abnormal increased to 4,642 (2,302+2,340) anaphase figures. This augmentation is critical in training deep learning method due to significant intrinsic imbalance between normal and abnormal anaphases in whole slide image.

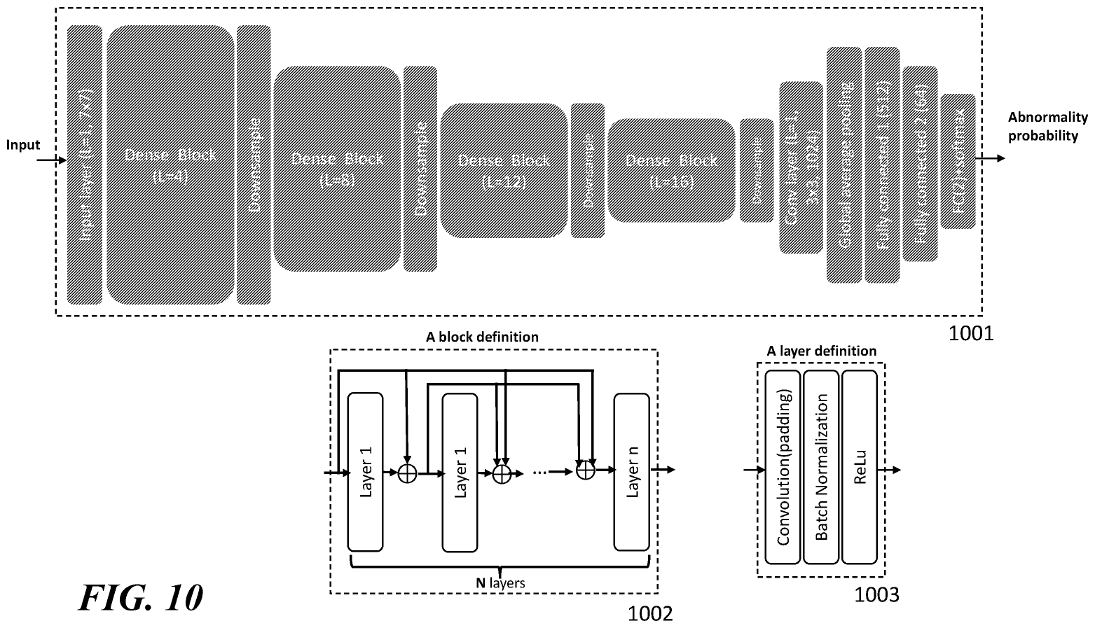

A custom DenseNet classifier was designed to automatically classify normal and abnormal anaphases. The input layer included 16 filters with a grow rate of 16. It includes 4 Dense blocks containing 4, 8, 12, 16 layers (N; 1002) (Convolution+Batch Normalization+ReLu (1003)). A Global Average Pooling (GAP), followed by two fully connected layers and softmax, were used as final layers. An Adam optimizer with learning rate of 0.0001 was used with cross entropy loss. GAP was used to handle different input image sizes. Model architecture is described in FIG. 10. The model outputs the probability that an anaphase event is abnormal (1001).

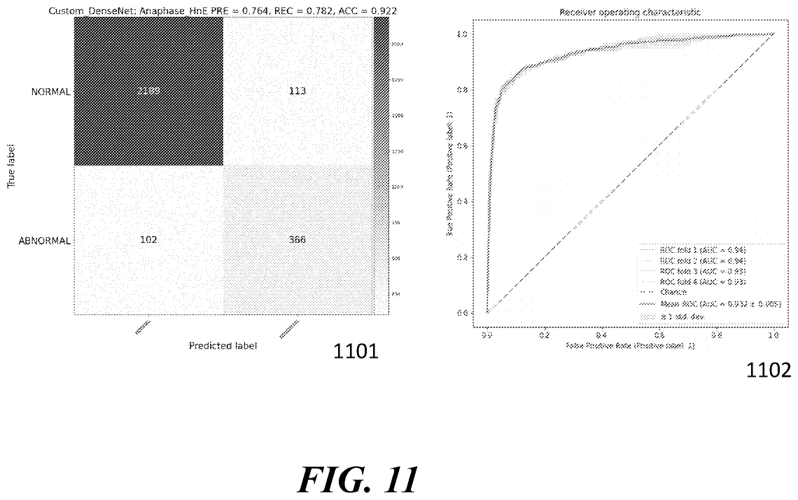

The trained model was used to classify the mouse mitotic events. The results of a 4-fold cross-validation study is shown in FIG. 11. The model performed with a precision of 0.764, recall of 0.782, and an accuracy of 0.922. The model correctly labeled 2,189 of 2,291 mitotic events as normal and 336 of 479 mitotic events as abnormal, thereby demonstrating the utility of the machine-learning model.

### Example 4: Anaphase Figure Detection Using a Point-Based Detection Network from Pre-Clinical Mice Images

This example describes the training and use of a machine-learning model provided herein for the detection of anaphase figures.

Pre-clinical mouse data was used to address the issue of human cancer lacking a sufficient number of anaphase events in H&E images, for training a deep learning model. 119 whole slide images of 12 different mouse syngeneic primary tumor models were used for training the deep learning model.

Whole slide images of mouse tumors were annotated by crowd-sourcing. 40 whole slide images of a mouse dataset were tiled to generate 19,022 1024×1024 histological image tiles. The 19,022 tiles were annotated by crowd-sourcing by drawing bounding boxes around anaphase figures within the images. From the 19.022 tiles, 3,437 tiles were identified by crowd sourcing to contain at least one anaphase figure, while the remaining 15.585 tiles did not contain any anaphase figures. In total, 4,220 anaphase events from the 3,437 tiles were identified.

For preparing images for training the deep learning model, 1024×1024 images were tiled to generate four 512×512 images, in order to provide enough resolution for the deep network to identify anaphase events. After tiling images to tiles of 512×512, there were 3,799 512×512 images that contained anaphase events and 72.049 512×512 images without any anaphase events. Data augmentation was used to increase the number of tiles with anaphase events to be used as training data. The data augmentation strategies included rotating, distorting, and zooming-in, and were applied to the abnormal anaphase events to increase the number of abnormal events. Also, in order to keep the balance between tiles with and without anaphase events, a subset of images without anaphase were selected in each epoch.

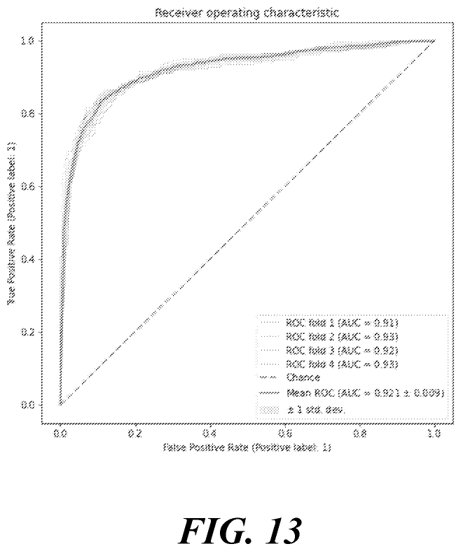

A CenterNet++ method with backbone of ResNet50 was used as an object detection model to detect anaphase figures in the histological image tiles, as described in FIG. 12. Results of a 4-fold cross-validation study for detection of anaphase figures using the CenterNet++ method are shown in FIG. 13 (each fold included the same number of images with and without anaphase events). The model performed with a recall (TPR) of 88%, an accuracy of 92%, and a precision of 65%, illustrating the metrics of the CenterNet++ method for detection of anaphase figures.

### Example 5: Exemplary Results of Experiments Involving Mitosis Detection in Computational Pathology

### Experimental Set-Up

### A. Dataset

Image magnification and tile sizes of TCGA BRCA were chosen, respectively, in consultation with expert labelers and based on an initial user experience experiment. 40× magnification and a tile size of 1000×1000 pixels were selected to best view mitotic events and minimize zooming, respectively. Using these specifications, each WSI in this study has approximately 800 to 3000 1k×1k tiles. The variance may be due to the differing sizes of the biopsy tissues.

### B. Labels

Two groups of labelers were used to gather data for our experiments: a group of five pathology experts and a crowd-sourced group of labelers with experience in medical domains.

The expert group completed two experiments: (1) placing bounding boxes indicating mitotic events on tiles and (2) labeling tiles with binary indicators for the presence or absence of mitosis. We refer to this data as “expert data” throughout. Expert data is aggregated by taking the majority to determine if mitosis present and then averaging the annotations [x1, y1,w, h] if the majority agrees mitosis is present. The results of the first experiment are used as the testing data and the results of both experiments are used to compare the time, accuracy, and concordance of the two strategies.

The tiles used in the expert labeler experiment were required to meet two selection criteria, chosen to increase the likelihood of discovering mitotic events. First, only high-grade TCGA BRCA WSIs were chosen as high-grade cancer suggests more mitotic events than lower grade. Second, from those WSIs, tiles were selected by applying an existing mitotic classification model (Dusenberry and Hu, 2018, referred to as IBM model) to all tiles and then picking the top tiles ranked by the confidence score output. The expert labeler dataset consists of 5 WSIs, with 800 tiles flagged with a high likelihood to contain a mitotic event, for a total dataset size of 4000 tiles.

The crowd-sourced group completed one experiment, placing bounding boxes indicating mitotic events on tiles (hereinafter referred to as “crowd-sourced data”). For this experiment, bounding boxes were only included in the dataset if at least five labelers have reviewed the tile and at least two labelers have placed overlapping boxes. An agreement score, a ∈[0, 1], is reported along with each box to capture this information. This data constituted the primary training data.

The input to the crowd-sourced data experiment consists of 70 patient BRCA WSIs with various cancer grades and a total of 56260 1k×1k tiles. For ten of these 70 WSIs, all non-background tiles were chosen. For the remaining WSIs, all tiles with an IBM model confidence score greater than 0.2 and an additional ten percent of tiles with score less than 0.2 were randomly chosen.

### C. Models

Five models were trained using different combinations of input data and prediction tasks (Table 1).

TABLE 1
Summary description of the trained model classes.

| Model | | Negative | |
|---|---|---|---|
| Class | Training Label | Tiles | Output |
| BC | c ∈ {0, 1} | ✓ | ĉ ∈ [0, 1] |
| Reg | a ∈ [symbol] _{>0} | | â ∈ [symbol] |
| Reg-NS | a ∈ [symbol] _{≥0} | ✓ | â ∈ [symbol] |
| Det | b ∈ [x₁, y₁, w, h] | | \hat{b} ∈ [x₁, y₁, w, h] |
| Det-WL | b ∈ [x₁, y₁, w, h], a ∈ [symbol] _{>0} | | \hat{b} ∈ [x₁, y₁, w, h] |

Note that all models take 250 × 250 pixel tiles as input.

Specifically, the following combinations were selected: (1) binary classification (BC), (2) regression with presence-only agreement score data (Reg), (3) regression with agreement score and negative samples (Reg-NS), (4) object detection (Det), and (5) object detection with weighted loss (Det-WL). Models 1-4 are straightforward following the descriptions herein. Model 5 alters the typical crossentropy loss for the output object detection network into a weighted cross-entropy where the weight is the corresponding agreement score.

### D. Metrics for Evaluation

To enable head-to-head comparisons of the different models, the true positive rate (TPR) and false positive rate (FPR) were selected as primary metrics of interest. To do this, the predictions from the object detection model were coarsened by treating each tile with any number predicted boxes as positive and each tile with no predicted boxes as negative.

### E. Model Training

### (i) Data Post-Processing

In all cases, for model training the 1k×1k pixel tiles were further tiled into 250×250 pixel tiles. This choice was motivated by the observation that the height and width each mitotic event is typically 30-60 pixels. In choosing a smaller tile size, the object of interest is a larger fraction of the overall image. A step size of 125 pixels was used, such that each tile overlaps with its neighbors. Using this procedure each 1k×1k pixel tile results in 49 250×250 pixel sub-tiles. Training, validation, and testing data splits are done respecting the source WSI such that one patient's data does not span the split.

### (ii) Label Post-Processing

Although the goal is to explore the interactions between data collection and model through classification, regression, and object detection in the context of mitotic detection, collecting each of these datasets can be cost prohibitive. Therefore, the most detailed data was gathered, bounding boxes, and applied coarsening techniques to use the data in the classification and regression settings. Specifically for the regression task the agreement score (a) of the tile, weighted by the fraction (p) of the bounding box contained in the tile was used, combined using a weighted sum in the case of multiple events (Equation 1):

$${s}_{t} = {\sum }_{i = 1}^{N} {p}_{i} \cdot {a}_{i} where p , a ∈ [ 0 , 1 ] . \qquad Equation 1$$

These scores are used directly for the regression task. For the classification task, tiles were assigned with a non-zero agreement score to be in the mitotic class and those with a zero agreement score were classified as not mitotic.

(iii) Training Setup

The training data can be described on the basis of unique mitotic events and tiles without any mitotic events (negative samples). Because of the nature of the problem, many tiles do not have any mitotic events and due to the sub-tiling strategy, most mitotic events appear on multiple tiles. Therefore, a unique identifier was assigned to each mitotic event. To obtain a data point (tile) from the dataset, a mitotic event ID was first sampled. Then a tile from the pool of tiles that contain the sampled mitotic event was sampled. Using this two-step process, the number of mitotic events used during training was defined. Object detection models were trained without negative samples (0:1 ratio), binary classification models were trained with negative samples (1:1 ratio), and regression models were trained with various ratios (0:1 to 1:1 ratios).

Briefly, based on initial tuning experiments, for thebBC, Reg, and Reg-NS models, the following parameters were chosen: batch size of 32, learning rate of 0.005, and the Adam optimizer (Kingma and Ba (2014)) with a step learning rate scheduler (γ=0.1 and step size of 15) was used. The model was trained for 60 epochs. For the Det and Det-WL, the following parameters were chosen: batch size of 16, learning rate of 0.005, and the SGD optimizer with a step learning rate scheduler (γ=0.1 and step size of 20) were used. Models were trained for 30 epochs. For all models, during training, a random horizontal flip and random photo-metric distortion from torchvision helper function was applied as image augmentation. For BC, Reg, and Reg-NS, the model started with torchvision's pre-trained ResNet50 and with a modified last layer. For BC, Reg, and Reg-NS, the best model was saved when the loss is lowest on the validation set. For Det and Det-WL, the pre-trained fastrenn-resnet50-fpn model with built-in object detection helper functions to evaluate trained model on validation set was used, and the model with the best performance was saved. The use of validation data containing negative samples for the selection of detection models (Det and Det-WL) was also explored, but it was determined that this decreased performance.

All models were implemented in PyTorch (Paszke et al. (2019)) using torchvision.

### Results

### A. Labeling Results

The labeled data are summarized in Table 2. In the expert data, only 506 out of 4000 tiles were mitotic for a total of 612 mitotic events. In the crowd-sourced data, 34987 out of 56260 tiles were found mitotic with a total of 52687 mitotic events.

TABLE 2
Labeled data summary

| | | | # Mitotic |
|---|---|---|---|
| | Type | # WSI | Events |
| Train | Crowd | 61 | 42203 |
| Validation | Crowd | 9 | 10484 |
| Test | Expert | 5 | 612 |

Table 3 compares the two labeling tasks amongst the expert labelers. In both settings, moderate concordance is achieved, although concordance is higher in the binary task, as measured by Fleiss kappa.

TABLE 3
Label acquisition comparison

| | Fleiss | Time | Mean |
|---|---|---|---|
| | Kappa | (sec) | Accuracy |
| Binary | 0.709 | 2.7 | 89% |
| Classification | | | |
| Bounding Box | 0.618 | 39 | 88% |
| Placement | | | |

Unsurprisingly, tile-level annotations are faster to acquire. Once tile size is accounted for, the time to acquire the same number of annotated pixels is approximately the same: there are 16 non-overlapping 250×250 pixel tiles per each 1k×1k tile and 2.7 sec×16≈40 sec. However, the diversity of tile-level annotations collected can be greater by selecting a more spatially diverse set. Accuracy is also reported, where ground truth is based on the majority expert vote, and find no appreciable difference in the two tasks. As the crowd-sourced labelers have less experience, we generally expect their labels to have more errors.

In summary, the rate of mitotic events per WSI was much higher among crowd-sourced data.

To assess the quality of the crowd-source label results, the experts to assessed the predictions. 2000 tiles were randomly selected from the crowd-sourced data, and the experts either confirmed or rejected the box, as well as added any missing boxes. The experts placed no new boxes and confirmed 80% of the bounding boxes.

### B. Modeling Results

The test results for the five model classes are summarized in Table 4.

TABLE 4
Summary of model performance on the test dataset.

| | True Positive | False Positive |
|---|---|---|
| Model | Rate % ↑ | Rate % ↓ |
| BC (0.5) | 91.4 | 40.8 |
| Reg (0.6) | 75.8 | 53.3 |
| Reg-NS (0.4) | 86.7 | 26 |
| Det | 90.6 | 30.6 |
| Det-WL | 93.2 | 43.5 |

The numbers in parentheses represent the prediction threshold, where relevant.

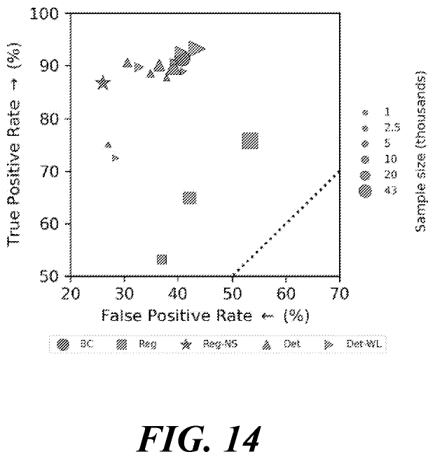

The object detection model with weighted loss (Det-WL) achieved the highest true positive rate and the regression with negative sampling (Reg-NS) achieved the lowest false positive rate. When looking at how the performance changes as a function of dataset size, we generally observe both TPR and FPR increasing together as dataset size increases (FIG. 14).

Depending on the particular setting, the relative importance of the TPR and FPR performance will vary. The expert data testing set has a large class imbalance, as is expected. The dataset consists of 19,600 250×250 tiles: 1,835 tiles have at least one mitotic event and 194,165 tiles have no mitotic events. Rather small changes in the FPR greatly change the number of tiles that are predicted positive.

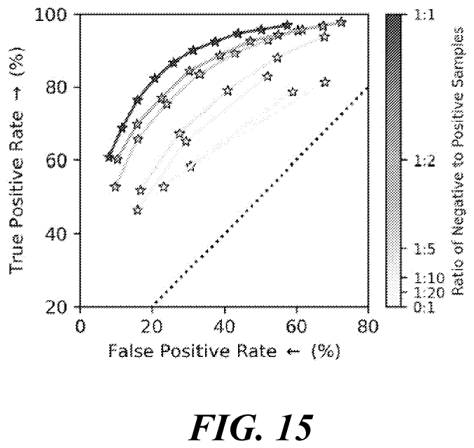

As described above, to use the classification and regression models in the context of detection, a threshold can be set. The impact of this threshold was investigated, along with different levels of negatives samples, on performance (FIG. 15).

For Det and Det-WL models, training was additional explored by loading the ResNet50 backbone obtained from the regression task to see whether a regression task model could improve the performance of the detection model. However, the performance was not close to the normal setting starting from PyTorch pre-trained weights on ImageNet. Even though the images for regression model and detection model are all digital pathology images, the number of samples used to train regression model is far from the ImageNet dataset. Thus, pre-training the ResNet50 backbone on digital pathology images did not outperform the ImageNet pretrained ResNet50.

Comparisons of aggregating all tiles with mitotic events into the training dataset was performed, in contrast to the sampling strategy applied, and found similar to worse performance depending on the exact setting. However in all settings, the naïve aggregation greatly increased the computational cost.

1. A method for characterizing a disease in a patient, comprising:
inputting one or more input histological images of a biological sample from a patient into a trained machine-learning model, wherein the trained machine-learning model is trained using a plurality of annotated training histological images;
identifying one or more mitotic events in the one or more input histological images using the trained machine-learning model, wherein the one or more identified mitotic events can be normal or abnormal mitotic events;
determining a frequency of abnormal mitotic events in the one or more input histological images based on the one or more identified mitotic events; and
classifying a pathological status of the biological sample based on the determined frequency of abnormal mitotic events in the one or more input histological images.

2. The method of claim 1, wherein the biological sample comprises at least a portion of a solid tumor.

3. (canceled)

4. The method of claim 1, wherein the plurality of annotated training histological images are from a biological sample from the patient.

5. The method of claim 1, wherein the one or more input histological images and/or the plurality of annotated training histological images:
(i) is captured at a resolution between 256 pixel×256 pixel and 10,000 pixel×10,000 pixel;
(ii) is captured between 20× and 100× magnification; and/or
(iii) is hematoxylin and eosin (H&E) stained.

6-7. (canceled)

8. The method of claim 1, further comprising segmenting one or more whole images into a plurality of image tiles, wherein the plurality of image tiles are used as the one or more input histological images and/or the plurality of annotated training histological images.

9. The method of claim 1, wherein the machine-learning model segments one or more the input histological images and/or the plurality of annotated training histological images into image tiles.

10. (canceled)

11. The method of claim 1, wherein the one or more input histological images and/or the plurality of annotated training histological images are processed to remove non-tumor tissue.

12. The method of claim 1, wherein the plurality of annotated training histological images are annotated by an individual or a plurality of individuals.

13. (canceled)

14. The method of claim 12, further comprising selecting a set of high confidence annotated training histological images from the plurality of annotated training histological images, wherein the set of high confidence annotated training histological images are selected based on concordance between annotations performed by a plurality of individuals.

15. (canceled)

16. The method of claim 1, wherein the plurality of annotated training histological images include one or more negative instance annotated training histological images, and wherein the one or more negative instance annotated training histological images do not comprise a mitotic event.

17. (canceled)

18. The method of claim 1, wherein the trained machine-learning model is an unsupervised model, a weakly-supervised model, or a human-in-the-loop model.

19-20. (canceled)

21. The method of claim 1, wherein the trained machine-learning model applies a model selected from the group consisting of Support Vector Machines (SVM), Random Forests (RF), Artificial Neural Network (ANN), Convolutional Neural Network (CNN), K-means, ResNet, DenseNet, eXtreme Gradient Boosting (XGBoost), VGG, swin-transformer, Faster-RCNN, Mask-RCNN, and CentralNet++.

22. (canceled)

23. The method of claim 1, wherein the one or more identified mitotic events with confidence above a specified threshold are used to compute a chromosomal instability metric of the biological sample, and wherein the pathological status of the biological sample is based on the chromosomal instability metric.

24. The method of claim 23, wherein the chromosomal instability metric is:
(i) the frequency of abnormal mitotic events with confidence above a specified threshold compared to all mitotic events in an input histological image of the one or more input histological images; or
(ii) the frequency of abnormal mitotic events with confidence above a specified threshold compared to all mitotic events in an input histological image tile normalized by a tumor area metric, wherein the tumor area metric is the tumor area calculated using a tumor image segmentor.

25-26. (canceled)

27. The method of claim 1, wherein the trained machine-learning model outputs:
(i) a chromosome instability metric for each input histological image tile of an input histological image; or
(ii) a chromosome instability metric aggregated for all input histological image tiles of an input histological image.

28-30. (canceled)

31. The method of claim 1, wherein characterizing the disease comprises diagnosing the disease, informing a treatment strategy for the disease, evaluating the disease progression, predicting the disease prognosis, evaluating an effect of a treatment for the disease, and/or identifying a patient population for treatment of the disease, based on the pathological status of the biological sample.

32-36. (canceled)

37. The method of claim 1, wherein the disease is a cancer.

38. (canceled)

39. A system for characterizing a disease in a patient with machine-learning, comprising: one or more processors; a memory; and one or more programs with instructions for:
receiving data representing one or more input histological images of a biological sample from a patient;
identifying one or more mitotic events in the one or more input histological images using a trained machine-learning model, wherein the trained machine-learning model is trained using a plurality of annotated training histological images, wherein the one or more identified mitotic events can be normal or abnormal mitotic events;
determining a frequency of the abnormal mitotic events in the one or more input histological images based on the one or more identified mitotic events; and
classifying a pathological status of the biological sample based on the determined frequency of abnormal mitotic events in the one or more input histological images.

40-77. (canceled)

78. A method for training a machine-learning model to analyze histological images of biological samples, comprising:
annotating a plurality of training histological images, wherein the annotating comprises identifying both normal and abnormal mitotic events in each training histological image of the plurality of training histological images; and
training a machine-learning model based on the plurality of annotated training histological images, wherein the machine-learning model is configured to receive one or more input histological images of a biological sample from a patient and determine a pathological status of the biological sample based on a determined frequency of abnormal mitotic events in the one or more input histological images.

79-119. (canceled)

120. A system for training a machine-learning model to analyze histological images of biological samples comprising one or more processors, memory, and one or more applications stored in the memory that include instructions for:
receiving a plurality of annotated training histological images, wherein the plurality of annotated training histological images are annotated to identify both normal and abnormal mitotic events in each training histological image of the plurality of training histological images; and
training a machine-learning model based on the plurality of annotated training histological images, wherein the machine-learning model is configured to receive one or more input histological images of a biological sample from a patient and determine a pathological status of the biological sample based on a determined frequency of abnormal mitotic events in the one or more input histological images.

121-162. (canceled)

163. A non-transitory computer-readable storage medium storing one or more programs, the one or more programs comprising instructions, which when executed by one or more processors of an electronic device having a display, which when executed by an electric device cause the electronic device to:
receive one or more input histological images of a biological sample from a patient;
identify one or more mitotic events in the one or more input histological images using a trained machine-learning model, wherein the trained machine-learning model is trained using a plurality of annotated training histological images, wherein the one or more identified mitotic events can be normal or abnormal mitotic events;
determine a frequency of abnormal mitotic events in the one or more input histological images based on the identified one or more mitotic events; and
classify a pathological status of the biological sample based on the determined frequency of abnormal mitotic events in the one or more input histological images.

164-205. (canceled)
